(19) United States
(12) Patent Application Publication
(10) Pub. No.: US 2026/0283960 A1
(43) Pub. Date: Sep. 24, 2026

(54) METHOD OF DRYING A THERAPEUTIC PRODUCT

(71) Applicant: Spraying Systems Co., Wheaton, IL (US)

(72) Inventors: Joseph P. Szczap, Naperville, IL (US); Katherine Buzecky, Sugar Grove, IL (US); Jyothi Rangineni, Aurora, IL (US)

(73) Assignee: Spraying Systems Co., Wheaton, IL (US)

(21) Appl. No.: 19/085,859

(22) Filed: Mar. 20, 2025

(51) Int. Cl. A61K 9/16; A61K 38/26

(52) U.S. Cl. CPC A61K 9/1688; A61K 38/26

(57) ABSTRACT

Provided is a therapeutic powder and a method for drying a therapeutic powder. The method comprises providing a feed solution containing a purified therapeutic agent that is extracted into a solvent. The method comprises electrostatically spray drying the feed solution containing the purified therapeutic agent to remove the solvent to form the therapeutic powder.

### CROSS-REFERENCE TO RELATED APPLICATIONS

This patent application claims the benefit of U.S. Provisional Patent Application No. 63/567,899, filed Mar. 20, 2024, which is incorporated herein by reference.

### BACKGROUND

More and more new therapeutic drug modalities are being discovered and further developed. Many of these drug modalities belong to the class of peptides, proteins, vaccines, cell and gene therapy, and biotherapeutic agents. Biotherapeutic agents (sometimes referred to simply as “biotherapeutics” or “biologicals”) are conventionally known as drug therapy products formed, at least partially, by extracting or producing the active ingredient or substance from a biological source. For example, the preparation of medicinal products containing biotherapeutics can include the use of animal and plant cells, even genetically-engineered bacteria, yeast, and fungi, among other sources.

Examples of biotherapeutics include, but are by no means limited to, stem-cell therapies, tissue-engineered products, gene-editing and other gene therapy products, cytokines, antibodies, proteins, hormones, peptides, and so forth. Due to the high costs and time for regulatory approvals involved in producing biotherapeutic agents, many proteins, peptides, nucleic acids, etc., have recently been manufactured by chemical synthesis.

These new therapeutics are increasingly used to treat serious disease and, in some instances, prevent the onset of disease, illness, or infections. Conventional applications include treatments for conditions such as cancer and diabetes, among others. They can also be used for a variety of treatments, including, inter alia, treating chronic inflammatory diseases. There exists interest in methods of preparation of therapeutics to increase efficacy as well as large-scale production, long shelf life, and ambient temperature storage and transportation.

The process of manufacturing new therapeutics can be preventative and present high costs in respect to time and monetary expense. Spray drying has been used as a process in producing powders for some products, but it has not been fully satisfactory in many therapeutic applications. For example, such systems require high inlet temperatures that result in high outlet temperatures during the drying process, which risk degrading the therapeutic product. Thus, there remains a need to effectively provide a therapeutic product that is stable and viable.

It will be appreciated that this background description has been created to aid the reader and is not to be taken as an indication that any of the indicated problems were themselves appreciated in the art. While the described principles can, in some aspects and embodiments, alleviate the problems inherent in other systems, it will be appreciated that the scope of the protected innovation is defined by the attached claims and not by the ability of any disclosed feature to solve any specific problem noted herein.

### BRIEF SUMMARY

The present disclosure relates to a therapeutic powder and its preparation. Surprisingly and unexpectedly, the therapeutic powder can be prepared using an electrostatic spray drying process, which allows for efficient production methods and with less risk of degradation of the therapeutic agent than conventional spray drying techniques. Embodiments of the disclosure further allow for the preparation of such therapeutics with good purity while removing water and/or solvents during the process. A lower drying (inlet) temperature can be used in some embodiments (e.g., about 140° C. or less). As a result, the embodiments of the present disclosure allow for a more efficient and cost-effective product process, as well as a high quality, stable therapeutic powder product.

Thus, in one aspect, the method of drying a therapeutic powder comprises providing a feed solution containing a purified therapeutic agent that is extracted into a solvent. Further, the method comprises electrostatically spray drying the feed solution containing the purified therapeutic agent to remove the solvent to form the therapeutic powder.

In another aspect, the disclosure provides a therapeutic powder. The therapeutic powder is prepared by providing a feed solution containing a purified therapeutic agent that is extracted into a solvent. The feed solution comprising the therapeutic agent is electrostatically spray dried to remove the solvent to form the therapeutic powder.

Further and alternative aspects and features of the disclosed principles will be appreciated from the following detailed description and the accompanying drawings. Accordingly, it is to be understood that both the foregoing general description and the following detailed description are exemplary and explanatory only and do not restrict the scope of the appended claims.

### BRIEF DESCRIPTION OF THE DRAWINGS

FIG. 1 is a front elevational view of an illustrative electrostatic spray drying system in accordance with an embodiment of the disclosure.

FIG. 2A is a diagrammatic depiction of the electrostatic spray drying system shown in FIG. 1.

FIG. 2B is a diagrammatic depiction of an alternative electrostatic spray drying system shown in FIG. 1.

FIG. 3 is an enlarged vertical section of the water jacketed powder direction cone of the illustrated electrostatic spray drying system in accordance with an embodiment of the disclosure.

FIG. 4 is an enlarged front elevational view of the water jacketed connecting pipe for coupling between the powder direction cone and the powder separation plenum of the illustrated embodiment in accordance with an embodiment of the disclosure.

FIG. 5 is an enlarged vertical section of the electrostatic spray nozzle assembly of the illustrated electrostatic spray drying system in accordance with an embodiment of the disclosure.

FIGS. 6A and 6B illustrate an enlarged partial vertical section of the powder separation plenum of the illustrated electrostatic spray dryer system in accordance with an embodiment of the disclosure.

FIG. 7 is a schematic diagram of an exemplary electrostatic spray drying system in accordance with an embodiment of the disclosure.

FIG. 8A is a first cross-section view of an electrostatic spray nozzle assembly in accordance with an embodiment of the disclosure.

FIG. 8B is a second cross-section view of an electrostatic spray nozzle assembly in accordance with an embodiment of the disclosure.

FIG. 9 is a detail cross-section view of nozzle head section, including an induction ring, of the electrostatic spray nozzle assembly depicted in FIGS. 8A and 8B.

FIG. 10 is an exploded perspective view of the electrostatic spray nozzle assembly depicted in FIGS. 8A and 8B.

FIG. 11 is a cross-section view of a multi-nozzle spray assembly incorporating the electrostatic spray nozzle assembly depicted in FIGS. 8A and 8B.

FIG. 12 is a further cross-sectional view of an electrostatic spray nozzle assembly in accordance with an embodiment of the disclosure.

FIG. 13A is a flow diagram of a conventional spray drying process in accordance with embodiments of the disclosure.

FIG. 13B is a flow diagram of an electrostatic spray drying process in accordance with embodiments of the disclosure.

FIG. 14 is a side elevational view of the powder processing tower of the illustrated spray dryer system in accordance with an embodiment of the disclosure.

FIG. 15 is a vertical section of the powder processing tower shown in FIG. 14.

FIG. 16 is a bar graph demonstrating inlet drying temperature (measured in ° C., charted along the X-axis) versus residual acetonitrile (ACN) in bovine serum albumin (BSA) powders (measured in ppm, charted along the Y-axis) following laboratory scale experiments.

FIG. 17 is a bar graph demonstrating inlet drying temperature (measured in ° C., charted along the X-axis) versus residual acetonitrile (ACN) in bovine serum albumin (BSA) powders (measured in ppm, charted along the Y-axis) following production scale experiments.

It should be understood that the drawings are not necessarily to scale and that the disclosed embodiments are sometimes illustrated diagrammatically and in partial views. In certain instances, details which are not necessary for an understanding of this disclosure or which render other details difficult to perceive may have been omitted. It should be understood that this disclosure is not limited to the particular embodiments illustrated herein.

### DETAILED DESCRIPTION

Embodiments of the present disclosure provide methods of drying a therapeutic powder. Embodiments of the disclosure are predicated, at least in part, on the surprising and unexpected techniques of drying therapeutic powders using a low heat, electrostatic spray system. Generally, electrostatic spray drying (ESD) is a technique that uses an electrostatic charge to a feed solution in order to dry at lower temperatures than conventional spray drying. Suitable devices, methods, and systems for electrostatic spray drying are depicted in U.S. Pat. Nos. 10,286,411, 10,279,359, 10,399,098, and 10,933,343, each of which is hereby incorporated by reference.

As described herein, the inventors have discovered through the use of the electrostatic spray system, therapeutic powders can be produced with more efficiency, using lower temperatures, and with less risk of degradation of the product than traditional high heat spray drying systems. Surprisingly and unexpectedly, the inventors have found that electrostatic spray drying allows for an efficient drying process by continuously drying at temperatures that are not detrimental to the biotherapeutics and eliminating pre- and post-drying steps, such as evaporation, concentration, and milling. The stability of the biotherapeutics is not compromised and negatives effects such as denaturation and aggregation are beneficially reduced or avoided.

As discussed herein, the methods relate to producing therapeutic powders using electrostatic spray drying processes from liquid formulations of therapeutics (e.g., peptides) in solvent or solvent and/or water systems. The therapeutic powders are used in the formation of therapeutic drugs which have multiple applications such as, for example, peptide drugs (e.g., GLP-1 agonists) or other pharmaceutical drugs. Peptide drugs are a unique class of pharmaceutical agents composed of a series of well-ordered amino acids with a specific bio-function. Peptide drugs commonly act as hormones, growth factors, neurotransmitters, ion channel ligands, or anti-infective agents and represent a significant and growing area for the treatment of simple to complex medical conditions. In some embodiments, the therapeutic product is a small molecule pharmaceutical, as the term is understood in the art. In addition, the therapeutic product can be a biologic, in accordance with some embodiments of the present disclosure.

Furthermore, the methods discussed herein provide an electrostatic spray dryer system that can be effectively operated with lesser heating requirements and fewer operating steps, and hence, more economically. A related aspect of the present disclosure provides a spray dryer system of such type that is operable for effectively drying temperature sensitive therapeutic compounds.

The surprising and unexpected methods discussed herein have benefits over traditional heat spray drying. Traditional techniques are cost and time intensive. In addition, the use of high heat has deleterious effects on peptides and other therapeutic agents, which can, inter alia, become inactivated at higher temperatures, thereby degrading the product.

The use of lower temperatures, such as those described herein, involve less risk of damage that can be caused by reactivity of solvents with active components at higher temperatures and therefore, cause contamination of the active agent with impurities in the final product (e.g., the therapeutic agent). Therefore, the surprising use of lower temperatures, in combination with the other features of the recited method, results in a production of therapeutic powder with lower impurities, for example, peptide or other therapeutic drugs.

Furthermore, in some embodiments, due in part to the surprising use of lower temperatures in the electrostatic spray drying methods discussed herein, less solvent retention is exhibited by the final product powders. As a result, among other things, the impurities level in the finished material (e.g., therapeutic product) is lower than when traditional spray drying processes and techniques are used. In some embodiments, the solvent can be at least partially recovered from the electrostatic spray drying production system.

In this regard, the methods discussed herein are more efficient than traditional heat spray drying. In addition to the reduced need or use of solvent, in some embodiments, the methods further reduce or eliminate the need for a precondensing step and a milling step, which are steps before and after the electrostatic spray drying step. The streamlined steps of the process, in combination with the lower temperatures discussed herein, results in a more efficient and less cost intensive process.

The process of manufacturing therapeutics such as peptides or proteins can be by recombinant technology of chemical synthesis. To illustrate recombinant technology, in order to engineer cells to produce specific peptides or proteins needed for various therapeutic applications, a gene encoding the desired protein is transferred into what is referred to as a “production cell.” Once the production cell is successfully manipulated to produce the encoded peptide or protein, the cell is then encouraged to multiply. The encoded peptide or protein is then extracted and purified further downstream.

Further steps follow the general production methodology set forth herein. These steps include extracting the respective therapeutic protein from the “nursery” cells in order to isolate the active agents from undesired cell material and even other forms of cellular protein. Such purification steps can involve process chromatography, among other potential techniques.

By way of example, and not limitation, process chromatography is one such technique that promotes the separation, identification, and purification of components of a mixture. As a part of the technique, proteins, for example, can be identified and ultimately purified (i.e., isolated from the mixture) based on certain defining characteristics (e.g., shape, size, total charge, hydrophobic groups, and binding capacity). Separation techniques for isolating a chosen protein can include, for example, ion exchange, surface adsorption, partition, and size exclusion. In addition, further or alternative chromatography techniques can be employed, including those based on the stationary bed (e.g., column, thin layer, and paper chromatography). In embodiments of the present disclosure, the purification extraction is performed using chromatography.

Chemical synthesis of therapeutic products such as peptides, proteins or oligonucleotides can be achieved by various methods. For example, one such method is solid phase peptide synthesis where peptides are synthesized using a solid phase system, cleaved, isolated, purified and extracted into a solvent.

Once the therapeutic agent is purified, further extraction techniques can be performed to isolate the therapeutic agent from, e.g., a feed solution. One such technique is the use of spray drying. For example, conventionally, spray drying techniques involve spraying liquid slurries into a drying chamber, into which heated air is introduced to dry the liquid into powder. Generally, the slurry commonly includes a liquid (e.g., water), an ingredient (e.g., food, flavor, or pharmaceutical), and a carrier. During the drying process, the liquid is driven off (e.g., by evaporation), thereby leaving the respective ingredient in power form encapsulated within the respective carrier. Spray drying systems can also be used to produce powders that do not require encapsulation (e.g., various food products, additives, and chemicals).

Existing spray drying systems can be relatively massive in construction, comprising of drying towers several stories in height. As such, these systems reflect substantial investments in terms of facility size requirements, capital investment needs, and associated running expenses. Not only is the equipment itself a substantial capital investment, the facility in which it is used must be of sufficient size and design to house such equipment. Heating requirements for the drying medium also can be expensive.

Surprisingly and unexpectedly, the present disclosure provides a method of drying a therapeutic powder, wherein the method comprises providing a feed solution containing a purified therapeutic agent that is extracted into a solvent and electrostatically spray drying the purified feed solution to remove the solvent to form the therapeutic powder. As discussed herein, the therapeutic agent includes peptides, enzymes, nucleotides, oligonucleotides, gene therapy agents, vaccines, and/or oligonucleotides. In some embodiments, the therapeutic agent is a peptide.

In some embodiments, the peptide functions as a hormone, a growth factor, a neurotransmitter, an ion channel ligand, or an anti-infective agent. In some embodiments, the anti-infective agent is an antiviral agent. In some embodiments, the anti-infective agent is an antibiotic agent.

In some embodiments, the therapeutic is a glucagon-like peptide 1 (GLP-1) agonist. Examples of GLP-1 agonists include, for example, semaglutide, tirzepatide, dulaglutide, exenatide, liraglutide, albiglutide, lixisenatide, GSBR-1290 (a small molecule agonist of the GLP-1 receptor), and any combination thereof. These therapeutics mimic the action of a hormone called glucagon-like peptide 1 in order to stimulate the body to produce more insulin when blood sugar levels start to rise after someone eats. The extra insulin helps lower blood sugar levels.

In some embodiments, the GLP-1 agonist is also a GLP-2 receptor agonist. An example of such a dual agonist is dapiglutide. In some embodiments, the GLP-1 agonist is a glucagon/GLP-1 receptor dual agonist, e.g., for the treatment of obesity, MASH (metabolic dysfunction-associated steatohepatitis), and fibrosis. An example of a glucagon/GLP-1 receptor dual agonist is survodutide. A glucagon/GLP-1 receptor dual agonist can result in increased energy expenditure, reduced appetite, improved liver function, and/or lowered blood sugar levels.

In some embodiments, the biotherapeutic is an amylin analog (amylin receptor agonist), e.g., for treatment of weight loss and/or diabetes. Amylin receptor agonists are synthetic or naturally derived molecules that mimic the action of amylin, binding to its receptor and eliciting similar physiological responses. Amylin primarily functions to slow gastric emptying, inhibit glucagon secretion, and promote satiety, thereby contributing to reduced food intake and potential weight loss. This differs from GLP-1 agonists, which mimic incretin hormones produced in the gut to suppress appetite and regulate blood sugar. Examples of amylin analogs include petrelintide, cagrilintide, GUB014295 (an agonist that specifically activates amylin and calcitonin receptors), CagriSema (a combination of the GLP-1 component semaglutide with amylin analog cagrilintide), pramlintide, amycretin (a dual agonist, targeting both GLP-1 and amylin receptors,), ACCG-2671 (an oral small molecule dual amylin and calcitonin receptor agonist), or combinations thereof.

In some embodiments, the therapeutic is a sodium glucose cotransporter 2 (SGLT-2) inhibitor. SGLT-2 inhibitors lower blood sugar by causing the kidneys to remove sugar from the body through urine. Examples of SGLT-2 inhibitors include canagliflozin, ertugliflozin, bexaglifloxin, dapagliflozin, empagliflozin, and any combination thereof.

In embodiments of the present disclosure, the feed solution comprises at least 0.5 wt. % therapeutic agent in solvent solution.

The Food and Drug Administration (FDA) devised guidelines to divide the use of solvents into three risk-based designations: Class I, Class II, and Class III solvents, as set forth by the International Council for Harmonization (ICH) through its Q3C Guideline and the United States Pharmacopeia (USP) through its 467 general chapter. Class I solvents should not be employed in the manufacture of drug substances, excipients, and drug products because of their unacceptable toxicity or their deleterious environmental effect. Class II solvents are solvents to be limited in pharmaceutical products because of their inherent toxicity. Class III solvents are solvents with low toxic potential to humans, which mean no health-based exposure limits are needed. These designations guide the amount of residual solvent that can be considered safe in pharmaceutical drugs. Organized by the level of toxicity and/or associated deleterious environmental effects, Class I solvents are considered to be the most restricted, while the use of Class III solvents generally faces the least restrictions. In this respect, Class I solvents are considered most toxic to organic life, while Class III solvents are the least toxic on the organizational scale.

In embodiments, the solvent can contain any suitable Class I solvent according to FDA guidelines. Specifically, in accordance with some embodiments, the Class I solvent contains benzene, carbon tetrachloride, 1,2-dichloroethane, 1,1-dichloroethene, and/or 1,1,1-trichloroethane.

In embodiments, the solvent can contain any suitable Class II solvent according to FDA guidelines. Specifically, in accordance with some embodiments, the Class II solvent contains acetonitrile, chlorobenzene, chloroform, cyclohexane, 1,2-dichloroethene, 1,2-dimethoxyethane, n,n-dimethylacetamide, n,n-dimethylformamide, 1,4-dioxane, 2-ethoxyethanol, ethylene glycol, formamide, hexane, methanol, 2-methoxyethanol, methylbutylketone, methylcyclohexane, methylene chloride, n-methylpyrrolidone, nitromethane, pyridine, sulfolane, tetrahydrofuran, tetralin, toluene, trichloroethylene, and/or xylene.

In embodiments, the solvent can contain any suitable Class III solvent according to FDA guidelines. Specifically, in accordance with some embodiments, the Class III solvent contains acetic acid, heptane, acetone, isobutyl acetate, anisole, isopropyl acetate, 1-butanol, methyl acetate, 2-butanol, 3-methyl-1-butanol, butyl acetate, methylethylketone, tert-butylmethyl ether, methylisobutylketone, cumene, 2-methyl-1-propanol, dimethyl sulfoxide, pentane ethanol, 1-pentanol, ethyl acetate, 1-propanol, ethyl ether, 2-propanol, ethyl formate, propyl acetate, and/or formic acid.

In some embodiments of the present disclosure, the solvent can contain any suitable combination of Class I-III solvents according to FDA guidelines. Specifically, in accordance with some embodiments, the solvent can contain any suitable combination of water, ethanol, acetaldehyde, benzene, carbon tetrachloride, 1,2-dichloroethane, 1,1-dichloroethene, 1,1,1-trichloroethane, acetonitrile, chlorobenzene, chloroform, cyclohexane, 1,2-dichloroethene, 1,2-dimethoxyethane, n,n-dimethylacetamide, n,n-dimethylformamide, 1,4-dioxane, 2-ethoxyethanol, ethylene glycol, formamide, hexane, methanol, 2-methoxyethanol, methylbutylketone, methylcyclohexane, methylene chloride, n-methylpyrrolidone, nitromethane, pyridine, sulfolane, tetrahydrofuran, tetralin, toluene, trichloroethylene, xylene, acetic acid, heptane, acetone, isobutyl acetate, anisole, isopropyl acetate, 1-butanol, methyl acetate, 2-butanol, 3-methyl-1-butanol, butyl acetate, methylethylketone, tert-butylmethyl ether, methylisobutylketone, cumene, 2-methyl-1-propanol, dimethyl sulfoxide, pentane ethanol, 1-pentanol, ethyl acetate, 1-propanol, ethyl ether, 2-propanol, ethyl formate, propyl acetate, and/or formic acid. In some embodiments, the solvent contains acetonitrile and water.

The solvent can have any suitable viscosity. In some embodiments, the solvent has a viscosity of from 1 cP to 1500 cP, such as, e.g., from 25 cP to 1500 cP, from 50 cP to 1500 cP, from 75 cP to 1500 cP, from 100 cP to 1500 cP, from 125 cP to 1500 cP, from 150 cP to 1500 cP, from 175 cP to 1500 cP, from 200 cP to 1500 cP, from 225 cP to 1500 cP, from 250 cP to 1500 cP, from 275 cP to 1500 cP, from 300 cP to 1500 cP, from 325 cP to 1500 cP, from 350 cP to 1500 cP, from 375 cP to 1500 cP, from 400 cP to 1500 cP, from 425 cP to 1500 cP, from 450 cP to 1500 cP, from 475 cP to 1500 cP, from 500 cP to 1500 cP, from 600 cP to 1500 cP, from 700 cP to 1500 cP, from 800 cP to 1500 cP, from 900 cP to 1500 cP, from 1000 cP to 1500 cP, from 1100 cP to 1500 cP, from 1200 cP to 1500 cP, from 1300 cP to 1500 cP, from 1400 cP to 1500 cP, from 1 cP to 1400 cP, from 1 cP to 1300 cP, from 1 cP to 1200 cP, from 1 cP to 1100 cP, from 1 cP to 1000 cP, from 1 cP to 900 cP, from 1 cP to 800 cP, from 1 cP to 700 cP, from 1 cP to 600 cP, from 1 cP to 500 cP, from 1 cP to 475 cP, from 1 cP to 450 cP, from 1 cP to 425 cP, from 1 cP to 400 cP, from 1 cP to 375 cP, from 1 cP to 350 cP, from 1 cP to 325 cP, from 1 cP to 300 cP, from 1 cP to 275 cP, from 1 cP to 250 cP, from 1 cP to 225 cP, from 1 cP to 200 cP, from 1 cP to 175 cP, from 1 cP to 150 cP, from 1 cP to 125 cP, from 1 cP to 100 cP, from 1 cP to 75 cP, from 1 cP to 50 cP, or from 1 cP to 25 cP. In embodiments, the solvent has a viscosity of 25 cP.

In embodiments of the present disclosure, the therapeutic product is electrostatically spray dried in a drying chamber having an inlet temperature of below 150° C. For example, the inlet temperature is about 140° C. or below, about 135° C. or below, about 130° C. or below, about 125° C. or below, about 120° C. or below, about 115° C. or below, about 110° C. or below, about 105° C. or below, about 100° C. or below, about 95° C. or below, or about 90° C. or below, 80° C. or below, 70° C. or below, 60° C. or below, 50° C. or below, or 40° C. or below. In comparison, conventional spray drying systems have a much higher inlet temperature, typically about 150-250° C. or 180-230° C. As one of ordinary skill in the art will appreciate, feedrate is dependent on many factors, including but not limited to, solid content of incoming liquid and inlet temperatures.

The atomizing gas temperature of the electrostatic spray dry system may be used at ambient or heated conditions. In some embodiments, the atomizing gas temperature is from 40° C. to 150° C., such as from, e.g., 40° C. to 140° C., from 40° C. to 130° C., from 40° C. to 120° C., from 40° C. to 110° C., from 40° C. to 100° C., from 40° C. to 90° C., from 40° C. to 80° C., from 40° C. to 70° C., from 40° C. to 60° C., from 40° C. to 50° C., from 50° C. to 150° C., from 60° C. to 150° C., from 70° C. to 150° C., from 80° C. to 150° C., from 90° C. to 150° C., from 100° C. to 150° C., from 110° C. to 150° C., from 120° C. to 150° C., from 130° C. to 150° C., or from 140° C. to 150° C.

The electrostatic spray drying process applies a voltage to the spray droplets or incoming liquid feed, which typically is about 0.1 kV or more (e.g., about 0.5 kV or more, about 1 kV or more, about 2 kV or more, about 4 kV or more, about 5 kV or more, about 7 kV or more, about 9 kV or more, about 12 kV or more, or about 15 kV or more). The upper limit of the applied voltage typically is 30 kV and in some instances, the upper limit is 20 kV or more preferably 15 kV. In the drying process, the applied voltage can be either continuous or modulated between two or more different voltages, known as Pulsed Width Modulation (PWM). Any two or more applied voltages ranging between 0.1-30 kV (e.g., 0.5 kV and 1 kV, 1 kV and 5 kV; 5 kV and 15 kV) can be used for PWM to provide a desired effect, such as a particular agglomerate size.

Alternatively, or in addition, to PWM, the charge (positive or negative) of the applied voltage can be altered, as necessary. Without wish to be bound by any theory, it is believed that alternating the electrostatic charge can change the surface composition of the particle and/or the agglomeration properties. For example, an applied negative charge will allow more polar compounds to move towards the surface of the particle and non-polar compounds will remain near the core of the particle. Accordingly, a negative electrostatic charge typically is applied in the electrostatic spray dry process. In some embodiments, the applied voltage is modulated between two or more different voltages.

The electrostatic spray drying can use any suitable applied voltage. In embodiments, the electrostatic spray drying uses applied voltage of at least −30 kV, e.g., −30 kV to 30 kV, −30 kV to 20 kV, −30 kV to 15 kV, −30 kV to 10 kV, −30 kV to 5 kV, −30 kV to 0 kV, −30 kV to −5 kV, −30 kV to −10 kV, −30 kV to −15 kV, −30 kV to −20 kV, −30 kV to −25 kV, −25 kV to 30 kV, −20 kV to 30 kV, −15 kV to 30 kV, −10 kV to 30 kV, −5 kV to 30 kV, 0 kV to 30 kV, 5 kV to 30 kV, 10 kV to 30 kV, 15 kV to 30 kV, 20 kV to 30 kV, 25 kV to 30 kV, −25 kV to 0 kV, −20 kV to 0 kV, −15 kV to 0 kV, −10 kV to 0 kV, or −5 kV to 0 kV.

The therapeutic powder that is produced can be formulated into a variety of different dosage forms. For example, the therapeutic product can be formulated into enteral formulations (e.g., tablets, capsules, pellets, granules, etc.); parenteral formulations (e.g., liquids, injectables (including subcutaneous injectables), etc.); topical formulation (e.g., cream, ointment, gel, paste, powder, etc.). In some embodiments, the therapeutic product is formulated into the form of an injectable formulation (e.g., a GLP-1 injectable). In some embodiments, the method further comprises pressing the powder into the form of a tablet. In embodiments, the powder can be mixed with a gel in the form of a capsule, etc.

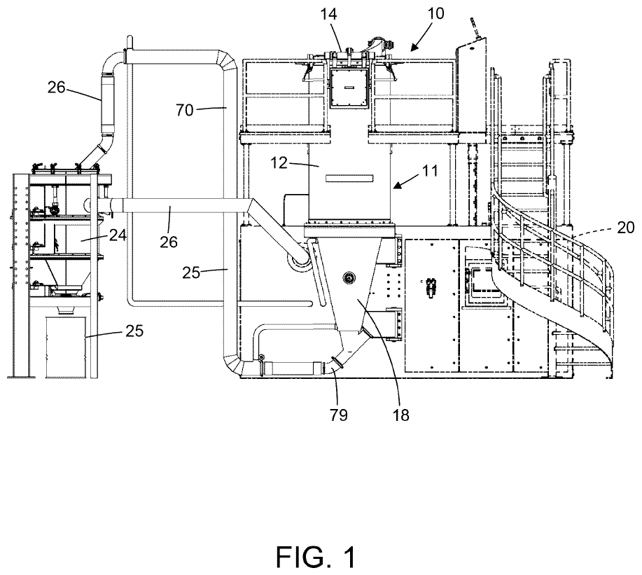

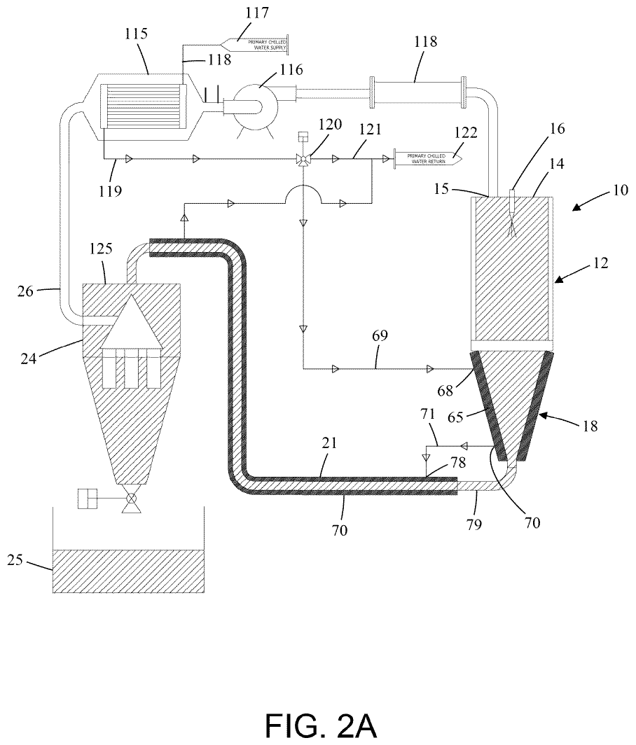

Turning now to the figures, particularly to FIGS. 1, 2A, and 2B, there is shown an illustrative electrostatic spray drying system 10 in accordance with the invention which includes a processing tower 11 comprising a drying chamber 12 in the form of an upstanding cylindrical body; a top closure arrangement in the form of a cover or lid 14 for the drying chamber 12 having a heating gas inlet 15 (FIGS. 2A and 2B) and an electrostatic liquid spray nozzle assembly 16; and a bottom closure arrangement in the form of a powder direction cone 18 supported at the bottom of the drying chamber 12. A frame supports the processing tower 11 in upright condition, which in this case includes stairs 20 for the operating personnel. A tube 21 communicates a drying gas and powder mixture from the powder direction cone 18 to a product separation plenum 24 supported in upright position by the frame adjacent the processing tower 11 for filtering powder from the drying gas and directing the dried powder into a powder receiving drum 25 and directing the filtered drying gas to return line 26.

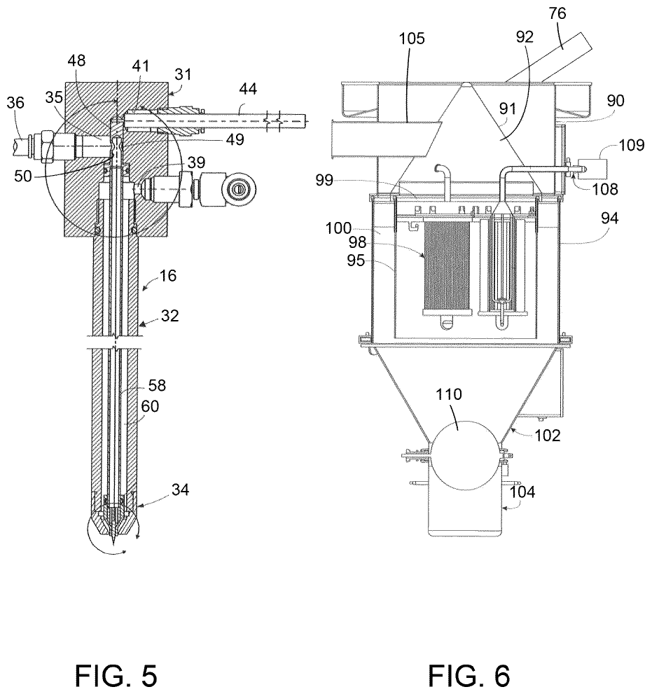

The drying chamber 12 and electrostatic spray nozzle assembly 16 may be of the type disclosed in U.S. Pat. No. 10,286,411 assigned to the same assignee as the present application, disclosure of which is incorporated herein by reference. The spray nozzle assembly 16, as depicted in FIG. 5, utilizes a pressurized air for directing a spray of electrostatically charged particles into the drying chamber 12 for quick and efficient drying of liquid slurries into desired powder form. The illustrated spray nozzle assembly 16 includes a nozzle supporting head 31, an elongated nozzle barrel or body 32 extending downstream from the head 31, and a discharge spray tip assembly 34 at a downstream end of the elongated nozzle body 32. The head 31 in this case is made of plastic or other non-conductive material and is formed with a radial liquid inlet passage 35 that communicates with a liquid supply line of 36 and in turn a supply of liquid to be dried.

The nozzle supporting head 31 in this case further is formed with a radial pressurized atomizing air inlet passage 39 downstream of said liquid inlet passage 35 that communicates with a suitable pressurized air supply. A radial passage 41 upstream of the liquid inlet passage 35 receives a high voltage cable 44 in abutting electrically contacting relation to an electrode 48 axially supported within the head 31 and extending downstream of the liquid inlet passage 35.

For enabling liquid passage to the spray tip assembly 34, the electrode 48 is formed with radial and axial passages 49, 50 communicating with a liquid feed tube 58, made of stainless steel or other electrically conductive metal, for communicating liquid from the axial electrode liquid passage 50 the downstream discharge spray tip assembly 34. An annular atomizing air passage 60 is defined between the liquid feed tube 58 and the outer cylindrical body 32 of the spray nozzle assembly 16 for directing atomizing gas from the atomizing air inlet 39 to the spray tip assembly 34 for atomizing the discharging liquid spray from the spray tip assembly 34. The cylindrical body of the drying chamber 12 preferably is made of a non-electrically conductive material and includes a removable and replaceable internal liner 21 for facilitating changeover between different liquids to be sprayed, again as disclosed in applicant's above referenced patent.

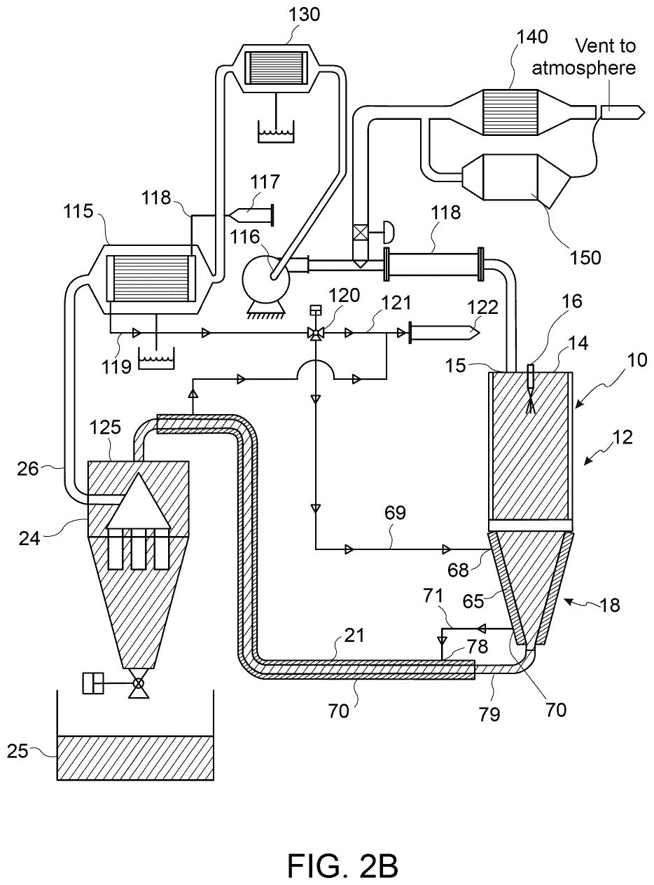

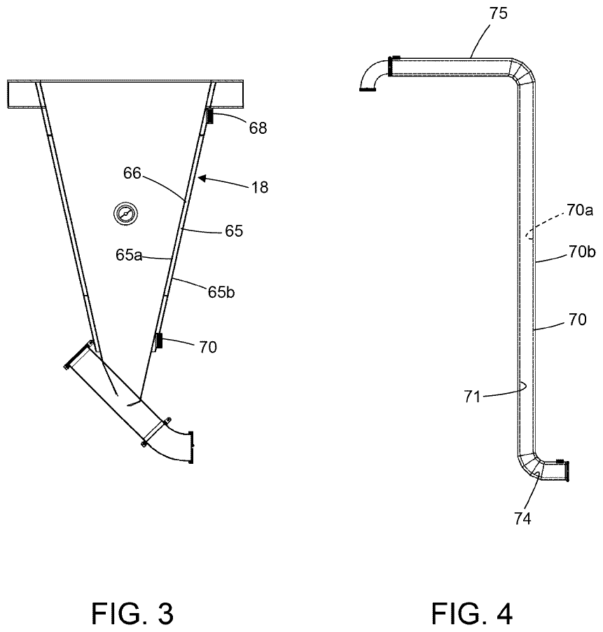

In carrying out an important aspect of the present embodiment, the powder direction cone 18 and the connecting tube 21 between the powder direction cone 18 and powder separation plenum 24 both comprise water jacket constructed heat exchangers for cooling the gas and entrained powder mixture immediately upon discharge from the drying chamber 12 for limiting exposure of the powder to damaging temperatures. The conical powder direction cone 18, which tapers inwardly in a downstream direction, is secured to the underside of the drying chamber 12 for directly receiving dried powder and heating gas from the drying chamber 12. The inwardly tapered conical wall of the powder direction cone 18 comprises a water jacket constructed heat exchanger 65 defined by inner and outer conical walls 65a, 65b which define a conical cooling water flow passage 66 (FIGS. 2A, 2B, and 3). The conical heat exchanger 65 in this case includes a cooling water inlet 68 adjacent an upper end thereof connected to a hose 69 coupled to a chilled water supply as will become apparent, and a cooling water return outlet 70 adjacent a lower end of the heat exchanger 65 coupled to an outlet hose 71. Chilled cooling water, preferably controlled at a temperature of between about 4.5° C. and 12.8° C. (i.e. 40°-55° F.) directed into the cooling water inlet 68 continuously flows through the flow passage 66 of conical heat exchanger 65 such that powder and drying gas directed into the powder direction cone 18 and contact with the heat exchanger 65 are immediately subjected to cooling upon discharge from the drying chamber 12.

In carrying out this embodiment, the connecting tube 21 between the powder direction cone 18 and the powder separation plenum 24 also comprises a water jacket constructed heat exchanger 70 having inner and outer walls 70a, 70b that define an annular cooling water flow passage 71, with the inner wall 70a being sized such that the gas will efficiently convey all entrained powder from the powder direction cone 18 to the powder separation plenum 24. The heat exchanger 70 in this case has an angled upstream end 74 coupled to an underside of the powder direction cone 18 and an upper angled end 75 connected to and communicating with an inlet 76 in an upper end of the powder separation plenum 24 for directing pre-cooled powder and drying gas into the plenum in a downward and tangential direction.

The connecting tube heat exchanger 70 has a chilled water inlet 78 coupled to a chilled water supply line 79 communicating from an outlet 81 adjacent a lower end of the powder collection cone 18 for receiving chilled water directly from powder direction cone heat exchanger 65. Hence, it will be seen that heating gas and powder discharging from the drying chamber 12 will come into substantial contact with the heat exchanging surfaces of the conical direction cone 18 and the connecting tube 21 for cooling before it is directed into the separation plenum 24. In practice, the heating gas and powder mixture discharging from the drying chamber at temperatures of 60° C.-90° C. can be cooled to temperatures of between about 25° C. and 50° C. (i.e., close to ambient temperature) prior to direction into the powder separation plenum 24. Such intermediate and substantial cooling has been found to prevent damage to even highly temperature sensitive powders.

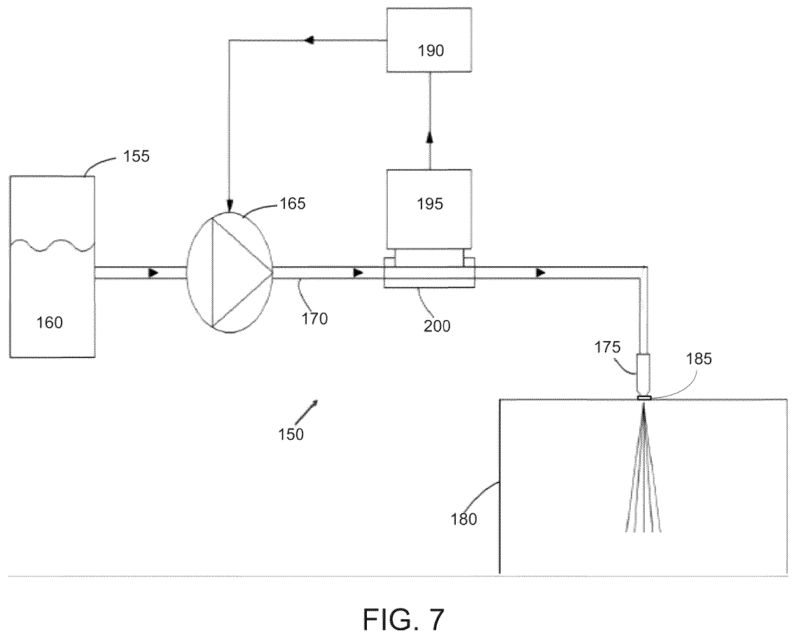

The illustrated powder separation plenum 24, as depicted in FIG. 6A, may be of a type depicted in applicant's above referenced patent, basically comprising an upper cylindrical plenum 90 having said powder receiving inlet 76; a downwardly opening conically configured exhaust plenum 91 disposed within said upper cylindrical plenum 90 that defines on its underside an exhaust chamber 92; a lower cylindrical plenum 94; a cylindrical filter element shroud 95 concentrically supported within the lower cylindrical plenum 94 within which a plurality of circumferentially spaced filters 98 (only one of which is shown in FIG. 6A) are supported in depending relation to a respective opening in a support plate 99.

During operation of the spray drying system 10, it will be seen that drying gas and powder directed into the powder direction plenum 24 will be channeled about the conical exhaust plenum 91 into the annular passageways 100 about the inner cylindrical filter element shroud 95 downwardly into a powder direction cone 102 for collection in a collecting chamber 104. While most of the dried powder remaining in the gas flow will migrate into the powder collection chamber 104, fine gas air borne particulate matter will be separated and retained by the annular filters 98 as the drying gas passes upwardly through the filters 98 into the drying gas exhaust plenum 91 for exit through a drying gas exhaust port 105 for recirculation to the drying chamber 12, as will be become apparent. For periodic cleaning of the filters 98 of buildup powder, the cylindrical filters 98 each have a respective reverse gas pulse cleaning device 108 cyclically operated by a control valve 109. A butterfly valve 110 in this case is mounted at an upper end of the collection chamber 104 and is operable for controlling the direction of powder into the collection chamber 104.

As seen in FIGS. 6A and 6B, for cleaning the cylindrical filters 98 of buildup of powder during the course of usage of the dryer system, the cylindrical filters 98 each have a respective reverse gas pulse cleaning device 108. To this end, a gas direction plenum in this case has an outer annular pressurized gas manifold channel 821 coupled to a suitable pressurized air supply. Each reverse air pulse cleaning device 108 has a respective pressurized gas supply line coupled between the annular pressurized gas manifold channel 821 and a respective control valve 109, which in this case mounted on an outer side of the air direction plenum. A gas pulse direction line or tube 828 extends from the control valve 109 radially through the air direction plenum and the conical wall of the exhaust plenum 91 and then with a right angle turn downwardly with a terminal discharge end 829 of the gas pulse directing line 828 disposed above and in aligned relation to a central opening of the filter cartridge holding plate 798 and underlying cylindrical filter element 796.

By appropriate selective or automated control of the control valve 109, the control valve can be cyclically operated to discharge pulses of the compressed gas from the line 828 axially into the cyclical filter 98 for dislodging accumulated powder on the exterior wall of the cylindrical filter element 796. The discharge end 829 of the pulse gas directing line 828 preferably is disposed in spaced relation to an upper end of the cyclical filter 98 to facilitate the direction of compressed gas impulses into the filter element 796 while simultaneously drawing in gas from the exhaust chamber 92 which facilitates reverse flow impulses that dislodge accumulated powder from the filter element 796. Preferably the discharge end 829 of the air tube 828 is spaced a distance away from the upper end of the cylindrical filter element such that the expanding air flow, depicted as 830 in FIG. 6B, upon reaching the filter cartridge, has an outer perimeter corresponding substantially to the diameter of the central opening in the cartridge holding plate 798. In the exemplary embodiment, the air direction tube has a diameter of about one inch and the discharge end 829 is spaced a distance of about two and a half inches from the holding plate 798.

The powder collection chamber 104 in this case has a circular butterfly valve 340 (shown in FIG. 6A in breakaway fashion within the powder collection chamber 104) mounted at an upper end of the collection chamber 104 operable by a suitable actuating device 841 for rotatable movement between a vertical or open position which allows dried powder to be directed into the collection chamber 104 and a horizontal closed position which blocks the passage of dried powder into the collection chamber 104 when powder is being removed. Alternatively, it will be understood that the powder collection chamber 104 could deposit powder directly onto a moveable conveyor from an open bottom end.

Surprisingly and unexpectedly, it has been found that increasing residence time in the spray chamber beneficially allows for reducing the residual solvent in the finished product. For example, closing the valve 340 will increase residence time. In some embodiments, the residence amount is from about 600 sec to about 1800 sec, such as from about 1800 sec to about 3600 sec. The control system allows operator to set the residence time on the material in the spray chamber. See, e.g., United States Patents U.S. Pat. No. 10,286,411B2, U.S. Pat. No. 11,033,914B2, U.S. Pat. No. 10,399,098B2, and U.S. Pat. No. 10,543,495B2, incorporated herein by reference.

The illustrated powder direction plenum 90 comprises an outer cylindrical housing wall 789 mountable in sealed relation to an underside of the drying chamber 12 and having an open upper end for receiving drying gas and powder from the drying chamber 12 and drying zone 1127. Housed within the powder direction plenum 90 is a downwardly opening conically configured exhaust plenum 91 which defines on its underside an exhaust chamber 92 (FIG. 6B) and on its upper side directs drying gas and powder from the drying chamber 12 downwardly and outwardly around an outer perimeter of the conical exhaust plenum 91.

The filter element housing 770 comprises an outer cylindrical housing wall 94 mounted in sealed relation by means of an annular seal 785 to a bottom peripheral edge of the powder direction plenum 90 and an inner cylindrical filter shroud 95 mounted in sealed relation by means of an annular seal 788 to the bottom peripheral edge of the conical exhaust plenum 91. The conical exhaust plenum 91 and the inner cylindrical filter shroud 95 are supported within an outer cylindrical housing wall 789 of the gas directing plenum 90 and filter element housing 770 by the plurality of radial supports so as to define air passageways 791 communicating about the bottom perimeter of the conical exhaust plenum 91 and an annular gas passageway 100 between the inner cylindrical filter shroud 95 and outer cylindrical housing wall 94 such that gas and powder passing through the powder direction plenum 90 is directed by the conical exhaust plenum 91 outwardly about the filter element shroud 91 into the underlying powder direction cone 102 and collection chamber 104.

The cylindrical filters 98 in this case are supported in depending relation to a circular support plate 99 fixedly disposed below the underside of the downwardly opening conical exhaust plenum 91. The circular filter support plate 99 in this case is mounted in slightly recessed relation to an upper perimeter of the cylindrical shroud 95 and defines a bottom wall of the exhaust chamber 92. The illustrated cylindrical filters 98 each are in cartridge form comprising a cylindrical filter element 796, an upper cylindrical cartridge holding plate 798, a bottom end cap and sealing plate 799 with interposed annular sealing elements. For securing the filter cartridges in assembled relation, the upper cartridge holding plate 798 has a depending U-shaped support member 804 with a threaded lower end stud positionable through a central aperture in the bottom end cap 799 which is secured by a nut 806 with a o-ring sealing ring 808 interposed therebetween. The upper holding plate 798 of each filter cartridge is fixed in sealed relation about a respective circular opening in the central support plate 99 with the filter element 796 disposed in depending relation to an underside of the support plate 99 and with a central opening in the holder plate 798 communicating between the exhaust chamber 92 and the inside of the cylindrical filter element 796. The filter element cartridges in this are disposed in circumferentially spaced relation about a center of the inner shroud 274.

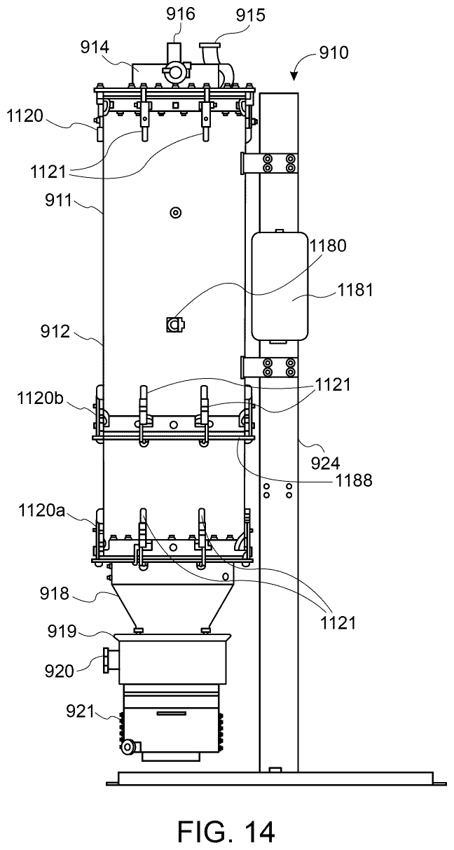

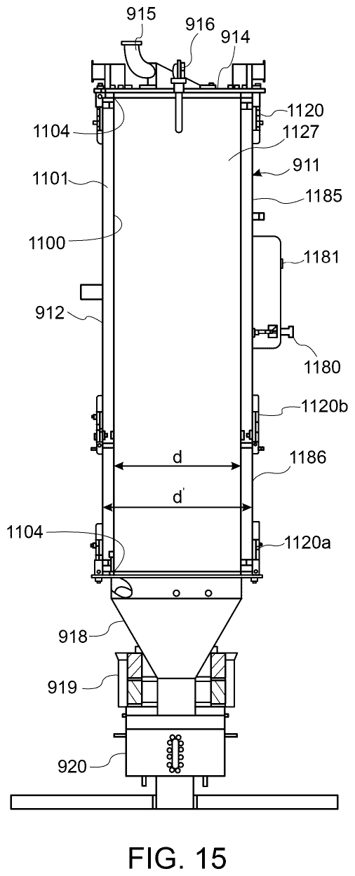

As seen in FIGS. 14 and 15, embodiments of an illustrative spray drying system 910 in accordance with the invention include a processing tower 911 comprising a drying chamber 912 in the form of an upstanding cylindrical structure, a top closure arrangement in the form of a cover or lid 914 for the drying chamber 912 having a heating air inlet 915 and a liquid spray nozzle assembly 916, and a bottom closure arrangement in the form of a powder collection cone 918 supported at the bottom of the drying chamber 912, a filter element housing 919 through which the powder collection cone 918 extends having a heating air exhaust outlet 920, and a bottom powder collection chamber 921. The drying chamber 912, collection cone 918, filter element housing 919, and powder collection chamber 921 all preferably are made of stainless steel. The top cover 914 preferably is made of plastic or other nonconductive material and in this case centrally supports the spray nozzle assembly 916. The illustrated heating air inlet 915 is oriented for directing heated air into the drying chamber 912 in a tangential swirling direction. A frame 924 supports the processing tower 911 in upright condition.

It will be appreciated that the drying gas introduced into the effective drying zone 1127 defined by the flexible liner 1100 both from the electrostatic spray nozzle assembly 916 and the drying gas inlet port 915, is a dry inert gas, i.e. nitrogen in the illustrated embodiment, that facilitates drying of the liquid particles sprayed into the drying chamber 912 by the electrostatic spray nozzle assembly 916. The recirculation of the inert drying gas, as described above, also purges oxygen from the drying gas so as to prevent the chance of a dangerous explosion of powder within the drying chamber in the event of an unintended spark from the electrostatic spray nozzle assembly 916 or other components of the system.

Pursuant to a further important feature of the present embodiment, the drying chamber 912 has an internal non-metallic insulating liner 1100 disposed in concentric spaced relation to an inside wall surface of the drying chamber 912 into which electrostatically charged liquid spray particles from the spray nozzle assembly 916 are discharged. As depicted in FIG. 15, the liner has a diameter d less than the internal diameter d1 of the drying chamber 912 so as to provide an insulating air spacing 1101, preferably at least about 2 inches (about 5 cm), with an outer wall surface of the drying chamber 912, but other dimensions may be used.

If during a drying process any particles may stick or otherwise accumulate on the surface of the liner 1100, a liner shaking device is provided for periodically imparting shaking movement to the liner 1100 sufficient to remove any accumulated powder. In the illustrated embodiment, the drying chamber 912 has a side pneumatic liner shake valve port 1180 which is coupled to a pneumatic tank 1181 that can be periodically actuated to direct pressurized air through the pneumatic liner shake valve port 1180 and into the annular air space between the liner 1100 and the outer wall of the drying chamber 912 that shakes the flexible liner 1100 back and forth with sufficient force to dislodge any accumulated powder. Pressurized air preferably is directed to the pneumatic liner shake valve port 1180 in a pulsating manner in order to accentuate such shaking motion. Alternatively, it will be understood that mechanical means could be used for shaking the liner 1100.

Surprisingly and unexpectedly, it has been found that shaking the liner reduces the buildup on the inside of the liner, increases batch powder production yield, reduces the amount of powder exposed to high heat in the upper portion of the chamber that could denature the material, reduces agglomerated product that builds up on the liner as droplets deposit repeatedly on material accumulated on the liner then fall off, and produces a particle size distribution. For example, in some embodiments the liner 1100 has a running state whereby a vacuum is pulled on the outside of the liner creating a cylinder shape inside the spray chamber from a flexible liner material. The liner is shaken as described in United States Patents U.S. Pat. No. 10,286,411B2, U.S. Pat. No. 11,033,914B2, U.S. Pat. No. 10,399,098B2, and U.S. Pat. No. 10,543,495B2, incorporated herein by reference. The liner shaker is turned on an off by the control system, cycle time and duration is set by the control system, and the liner shaker's pressure is set by the control system. The liner shaker cycle includes a period of time where the drying gas flow moves in a cyclonic fashion through the dryer, causing the liner to shake and providing additional removal of powder from the liner before the liner stabilizes in the running state.

In order to ensure against cross contamination between successive different selective usage of the spray dryer system, such as between runs of different powders in the drying chamber 912, the annular arrays 1120, 1120 a of quick disconnect fasteners 1121 enable disassembly of the cover 914 and collection cone 918 from the drying chamber 912 for easy replacement of the liner 1100. Since the liner 1100 is made of relatively inexpensive material preferably it is disposable between runs of different powders, with replacement of a new fresh replacement liner being affected without undue expense.

The drying chamber 912 has a modular construction that permits reducing the length of the drying chamber 912. In the illustrated embodiment, the drying chamber 912 comprises a plurality, in this case two, vertical stacked cylindrical drying chamber modules or sections 1185, 1186. The lower chamber section 1186 is shorter in length than the upper chamber section 1185. The two cylindrical drying chamber sections 1185, 1186 again are releasably secured together by an array of circumferentially spaced quick disconnect fasteners 1121. A mounting ring for the array of fasteners 1121 is welded to the upper cylindrical drying chamber section 1185 adjacent the lower end thereof and the fasteners 1121 of the array are oriented with draw hooks downwardly positioned for engaging and retaining an underside of a top outer radial flange 1188 (FIGS. 14 and 15) of the lower cylindrical drying chamber section 1186. Upon release of the arrays of fasteners 1121 affixing the lower cylindrical section 1186 to the upper cylindrical section 1185 and the collection cone 18, the lower cylindrical section 1186 can be removed, the lower standoff ring assembly 1104 repositioned adjacent the bottom of the upper chamber section 1185, and the liner 1100 replaced with a shorter length liner. The upper cylindrical dryer chamber section 185 can then be secured directly onto the powder collection cone 18 with the lower standoff ring assembly 1104 therebetween by the fasteners 1121 of the array then engage an outer annular flange of the collection cone 18. This modification enables use of a substantially shorter length effective drying zone for further reducing heating requirements for smaller lot drying.

For recirculating and reusing exiting drying gas from the separation plenum 24, the separation plenum outlet port 105 is coupled to the recirculation line 26 which in turn is connected to the heating gas inlet port 15 of the top cover 14 of the drying chamber 12 through a condenser 115, a blower 116, and a drying gas heater 118 (FIGS. 2A and 2B). The condenser 115 removes any water vapor from the exhaust gas flow stream by means of cold water chilled condensing coils 115a having respective cold water supply 117 and return lines 118, 119. The dried gas may then directed by the blower 116 through the gas heater 118 which reheats the drying gas after cooling in the condenser 115 to a predetermined heated temperature for the particular powder drying operating for redirection back to the heating gas inlet port 15 and into the drying chamber 12.

As depicted in FIGS. 2A and 2B, condenser 115 is used primarily for the removal of water vapor from the drying gas stream. Secondary chilling coil 130 is used primary for the removal of solvent from the drying gas steam. Primary vent condensing coil 140 is used to condense any remaining solvent before it is vented to atmosphere for pollution control. Secondary vent condensing coil 150 is used to condense any remaining solvent before it is vented to atmosphere when the primary vent condensing coil 140 has formed an ice barrier that restricts the venting of the drying gas.

Each housing of condenser 115, secondary chilling coil 130, primary vent condensing coil 140, and secondary vent condensing coil 150 collects condensate, either water, solvent, or a combination thereof and drains it into a collection container for recovery. The condensate is properly drained under gravity or pumped into the collection containers from a drain pan under the condensing coil.

Each housing of condenser 115, secondary chilling coil 130, primary vent condensing coil 140, and secondary vent condensing coil 150 uses a flow control valve that modulates the appropriate temperature chilled water (glycol/water combination) based on the reading from a temperature sensor placed on the exit side of the condensing coil. The control system allows the operator to set the proper coil exiting gas temperature.

Condenser 115 is just cold enough to remove water vapor from the drying gas stream without freezing the water vapor on the chilled water coil since this would restrict the drying gas flow.

Secondary chilling coil 130 is as cold as necessary to remove as much solvent vapor from the drying gas stream as possible without freezing any solvent or remnant water vapor not removed by condenser 115.

Primary vent condensing coil 140 and secondary vent condensing coil 150 are as cold as possible to remove as much solvent from the vent stream as possible before it is vented to atmosphere. It is likely that this temperature is so low that freezing and restriction of the coil is to be expected. So as primary vent condensing coil 140 is in a condensing mode, secondary vent condensing coil 150 is in a defrosting mode. Defrosting is performed by an external means to the coil such as a radiant heater, or internally by switching from cold water to hot water in a controlled manner so as not to shock the coil and cause rapid thermal expansion that could lead to premature failure of the coil. Switching the vent flow from primary vent condensing coil 140 to secondary vent condensing coil 150 is performed by valves in the vent stream.

In keeping with a further feature of the present embodiment, the chilled water exiting the condenser 115 may be recirculated, in whole or in part, to the water jacket constructed heat exchanger 65 of the powder direction cone 18. In the illustrated embodiment, the condenser 115 is coupled to a three-way diverting valve 120 enabling chilled water from the condenser 115 to be directed to the inlet 65 of the powder direction cone heat exchanger 65 or to a return line 121 to the water supply 122 for re-cooling. In further carrying out this feature, the position of the valve 120, and correspondingly the chilled water flow rates, are controlled on a temperature basis from a temperature sensor 125 located in the powder separation plenum. Hence, the temperature of the chilled water supply to the powder collection cone heat exchanger can be selectively controlled for predetermined cooling of the powder gas mixture exiting the drying chamber.

From the foregoing, it can be seen that the heat exchange features of the powder direction cone and connection tube between the powder direction cone and the separation plenum enable immediate cooling of gas and entrained powder mixture exiting the drying chamber well before it reaches the gas powder separation apparatus, hence limiting the exposure of the dried powder to damaging temperatures. The chilled water can be precisely controlled by upstream temperature sensing. The electrostatic spray drying combined with such immediate gas powder and gas cooling makes the system well suited for processing heat sensitive powders. The immediate product cooling results in maintained activity of the finished dried product, allowing use of the system on extremely heat sensitive formulations. Moreover, the production of dried powder can be improved substantially since the inlet temperature of the drying gas can be operated at temperatures that improve the evaporation rate in the drying chamber. Since the exposure time in that area is minimal, because the powder reaches the cooling section immediately, the powder can be produced at a substantially faster rate while still being protected.

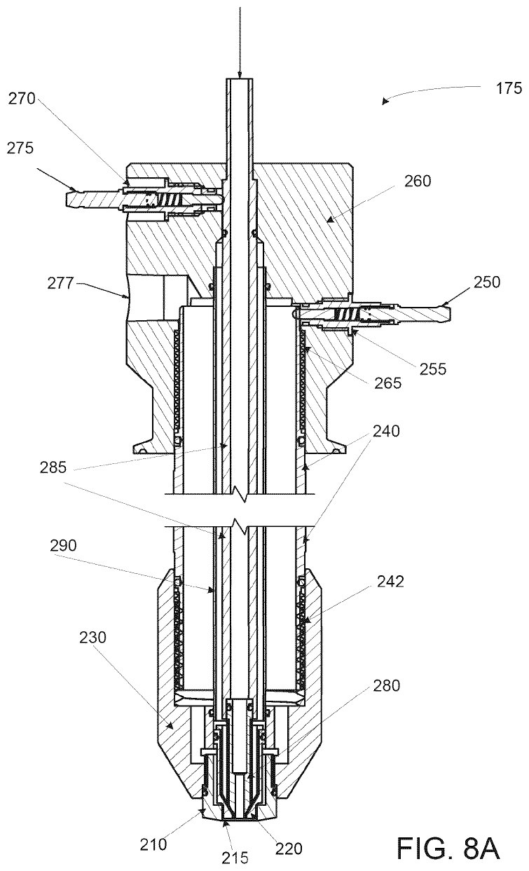

In the present disclosure, an arrangement is provided for providing an electrostatically charged spray nozzle that incorporates an electrical circuit arrangement that ensures proper operation of an electrostatic spray nozzle that includes an induction ring for providing an electrostatic charge to the output spray of the electrostatic spray nozzle. Turning to FIG. 7, an exemplary electrostatic spray drier system 150 is illustratively depicted. In the illustrative example, a tank 155 holds a liquid feed stock 160. The liquid feed stock 160 is drawn by a motor-driven pump 165 from the tank 155 into and through a feed line 170 for discharge at an electrostatic spray nozzle 175 inside a spray drying chamber 180. Importantly, the spray nozzle includes an induction ring 185. The induction ring 185 is positioned at an exit aperture of the electrostatic spray nozzle such that, in operation, the high voltage electric field generated by the induction ring (e.g., 3000 Volts) applies/establishes an electrostatic charge potential to droplets within a spray created from the liquid feed stock 160 discharged from the electrostatic spray nozzle 175.

An aspect of a particular configuration, of the electrostatic spray nozzle 175 including the induction ring 185, adapts/configures the electrostatic spray nozzle 175 for particular use in spray drying of a feedstock. In particular a sufficient voltage differential is applied, between the induction ring 185 and the feedstock at an exit aperture of the nozzle 175, to enhance droplet formation from the feedstock material by an induced charge present in the liquid passing from the exit aperture of the nozzle 175. Such voltage may be 3,000 to 4,000 volts, which is substantially lower (e.g. an order of magnitude) than known electrostatic spraying systems that operate at, for example, 30,000 volts. Additionally, given variations in conductivity of feedstock, a closed loop control arrangement is contemplated in illustrative examples to facilitate an automatic setting of a voltage difference between the induction ring 185 and the exit aperture of the electrostatic spray nozzle 175 to ensure sufficient voltage is applied to ensure enhanced/desired droplet formation without excessive voltage being applied. Such feedback arrangement can be carried out, for example, by incorporating an electrical current sensor in the induction circuit that senses both too little current (i.e. induction electrical field magnitude needs to be increased) and too much current (i.e. induction electrical field magnitude needs to be decreased).

A controlled liquid feed stock delivery system, including the pump 165, delivers the liquid feed stock 160 at a specified flow rate to the spray nozzle 175. The motor-driven pump 165 is controlled by a controller 190 (e.g., a programmable logic controller) in accordance with a specified set point and a currently sensed flow rate. An operator specifies, for example, a flow rate set point via a human-machine interface (HMI) and then activates the motor-driven pump 165. Thereafter, the controller 190 monitors (via sensor input signals) a flow rate of the liquid feed stock and adjusts (via motor control signals) motor speed of the motor-driven pump 165 to maintain a set/specified flow rate of the liquid feed stock 160 to the spray nozzle 175. In order to maintain a desired flow, the controller 190 continuously receives a measurement signal indicative of an instantaneous flow rate of the liquid feed stock passing through a feed line to the spray nozzle. An in-line flowmeter 195 measures instantaneous flow rate of the liquid feed stock 160 through a pipe section 200 to which the in-line flowmeter 195 is operationally mounted. The in-line flowmeter 195, in turn, provides a signal to the controller 190 that maintains a historical record of sensed flow and provides control over the overall operation of the electrostatic spray drier system 150 (including a speed of the motor-driven pump 175).

Details of the general structure of the electrostatic spray drying system 10, including the controller 190, are well known to those in the industry and thus are not discussed in detail herein. Rather, attention is directed to an exemplary electrical/structural arrangement of spray nozzle including an induction ring mounted proximate an exit aperture thereof for providing an electrostatically charted droplet stream of the liquid feedstock in accordance with illustrative examples of the present disclosure.

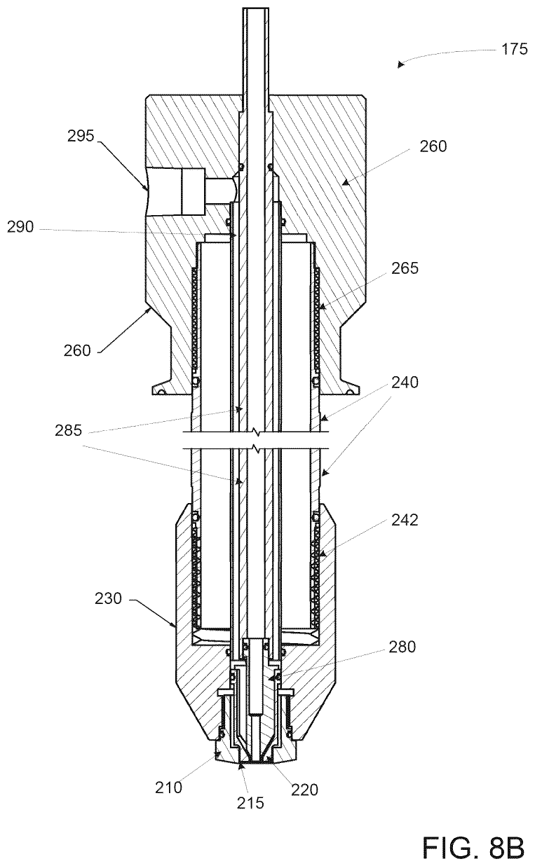

By way of a first specific example, the spray nozzle 175 is specially configured nozzle assembly that, in operation, exhibits certain electrical properties facilitating generation of a continuous flow of electrostatically charged spray droplets. Turning to FIG. 8A, an exemplary electrostatic spray nozzle arrangement is illustratively depicted where electrostatic charging of spray droplets is achieved by an electrical circuit arrangement including an induction ring 210 (corresponding to the induction ring 185 in FIG. 7) provided in the form of an electrically conductive metal retaining cap positioned at an exit aperture of the spray nozzle 175. An opening 215 of the induction ring 210 is sufficiently wide to avoid, with the aid of a purging gas stream, excessive buildup of the liquid feedstock emitted from an opening of an atomizing gas cap 220 that passes in droplet form through the opening 215. By way of example, the opening 215 has an inner diameter on the order of less than 1 inch for an applied electrical field having a voltage of 3,000 to 4,000 volts (3-4 kilovolts). More particularly, the opening 215 has a diameter of about 0.7 inches. However, in accordance with various spray drying applications, the diameter of the opening 215 and/or the applied voltage (electrical field potential between the induction ring 215 and liquid feedstock exiting the nozzle) are modified in accordance with spray pattern (wide/narrow spray field), nozzle aperture position (linear displacement along path of spray field) in relation to the opening 215 of the induction ring 210. In the illustrative example, the atomizing gas cap 220 is a non-conductive insulating material (e.g., a rigid plastic material).

A first conductive path is provided for generating an electrostatic field at the opening 215 of the induction ring 210 to electrostatically charge droplets of feedstock emitted from the atomizing gas cap. To that end, the induction ring 210 physically (by complementary screw threating) and conductively engages an electrically conductive surface of a nozzle head 230. The first conductive path is further provided by a further physical and conductive engagement of the nozzle head 230 with a purge gas tube 240. By way of example, the nozzle head 230 and the purge gas tube 240 are physically and conductively engaged by complementary screw thread surfaces at 242. The purge gas tube 240 is also provided with an electrically conductive surface providing an electrically conductive path from the nozzle head 230 to an induction field (high voltage) electrode 250 from a high voltage field signal source (not shown).

In an illustrative example, outer surfaces of electrically conductive components, (e.g., the induction ring 210) are coated with an electrically insulating layer to reduce the possibility of arcing within the spraying environment. As such, only the inner surface (or portion thereof) of the exposed surfaces of the induction ring 210 (as opposed to a non-exposed threaded surface of the induction ring 210 that is also a conductive surface) is a conductive surface. Such electrically insulating layer is provided by, for example, a polytetrafluoroethylene (PTFE) coating.

In yet a further illustrative example, all exposed surfaces of electrically conductive components—even the inner exposed surface of the induction ring 210—are coated with a strong dielectric material (e.g. PTFE) to provide an electrical insulating barrier between the high (magnitude) voltage of the induction ring 210 and low (magnitude) voltage of the feed stock as well as any potentially ground connection sources to which the feed stock comes into contact prior to exiting the spray nozzle. Such arrangement facilitates preventing, minimizing any current flow from the induction ring during operation of the illustrative electrostatic spray drying system.

A second conductive path is provided for establishing a complementary electrical (e.g., ground) path from conductive feed lines through which the feedstock passes from the tank 155 (see FIG. 7) to the atomizing gas cap 220. The second conductive path provides a source for inducing a charge (opposite the field potential generated at the opening 215) on the droplets passing from a fluid tip 280 having an electrically grounded conductive surface in contact with the feedstock) through an electric field at the opening 215. The second conductive path continues at a physical and electrical connection between the fluid tip 280 and a fluid tube 285 that provides the feedstock to the fluid tip 280. Similarly to the outer surface of the induction ring, the outer surfaces of the fluid tip 280 and fluid tube 285 are coated with an electrically insulating layer (e.g., PTFE).

An atomizing gas tube 290 provides atomizing gas to the atomizing gas cap 220. The atomizing gas tube 290 is, by way of example made of a non-electrically conductive material (e.g., a rigid plastic, ceramic, etc.) that is configured to provide a sealed engagement with the atomizing gas cap 220. Alternatively, the atomizing gas tube 290 comprises a conductive material coated with an electrically insulating material. As such, the atomizing gas tube 290 and atomizing gas cap 220 provide an electrically insulating barrier between the first conductive path and the second conductive path described herein above. It is noted that such electrically insulating characteristic may alternatively be achieved by coating exposed surfaces with an insulating coating (e.g., PTFE).

As shown in FIG. 8A, a nozzle body 260 is physically configured with several receptacles/openings for maintaining physical/electrical engagement between components of the spray nozzle 130 illustratively depicted herein. In the illustrative example, the nozzle body 260 includes an induction field electrode receptacle 255 holding the induction field electrode 250 in electrically conductive engagement with the electrically conductive surface of the purge gas tube 240. The nozzle body 260 includes a ground electrode receptacle 270 holding an electrical ground electrode 275 in electrically conductive engagement with the electrically conductive surface of the fluid tube 285. An induction ring purge gas port 277 provides an opening for feeding a purge gas that flows through the purge gas tube 240 to the opening 215 in the induction ring 210. As further shown in FIG. 8B (a further cross sectional view rotated 90 degrees from the view depicted in FIG. 8A), the nozzle body 260 further includes an atomizing gas port 295 that provides an opening for feeding an atomizing gas to the atomizing gas tube 290.

As shown in FIG. 8A, the nozzle body 260 includes a cylindrical receptacle having a threaded surface at 265 to hold in place the purge gas tube 240 having a complementary threaded outer surface.

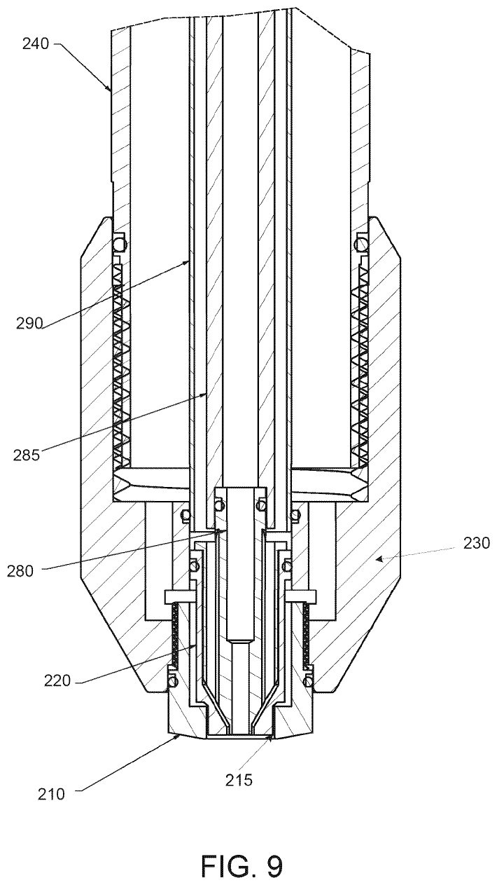

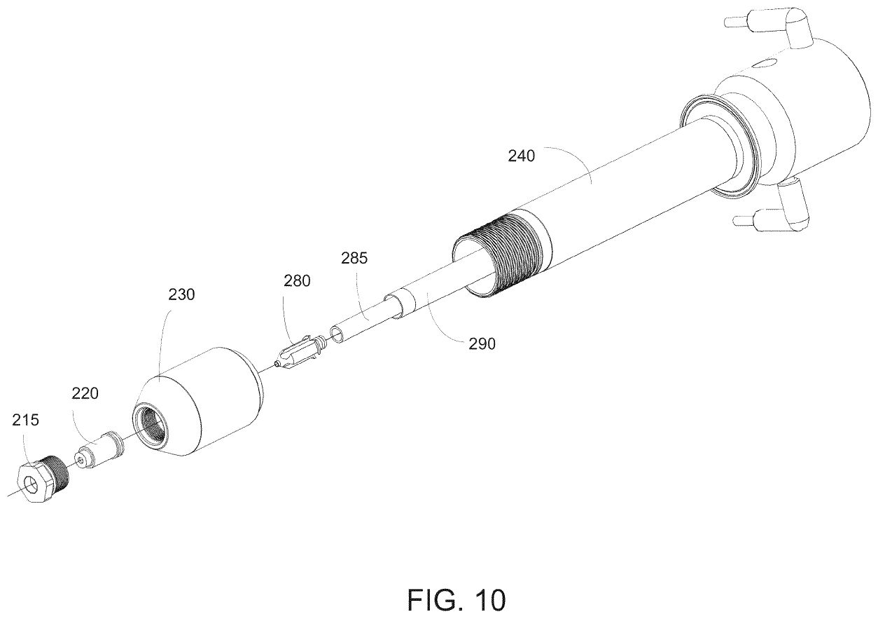

Turning to FIG. 9, an additional detailed view is provided of the nozzle head portion of the spray nozzle depicted in FIGS. 8A and 8B to enable a clearer view of the various physical relationships depicted in FIGS. 8A and 8B and the corresponding written description provided herein above. Additionally, FIG. 10 provides an exploded perspective view of the electrostatic spray nozzle assembly depicted in FIGS. 8A and 8B to provide additional visual details of the illustrative example of an electrostatic spray nozzle in accordance with the current disclosure.

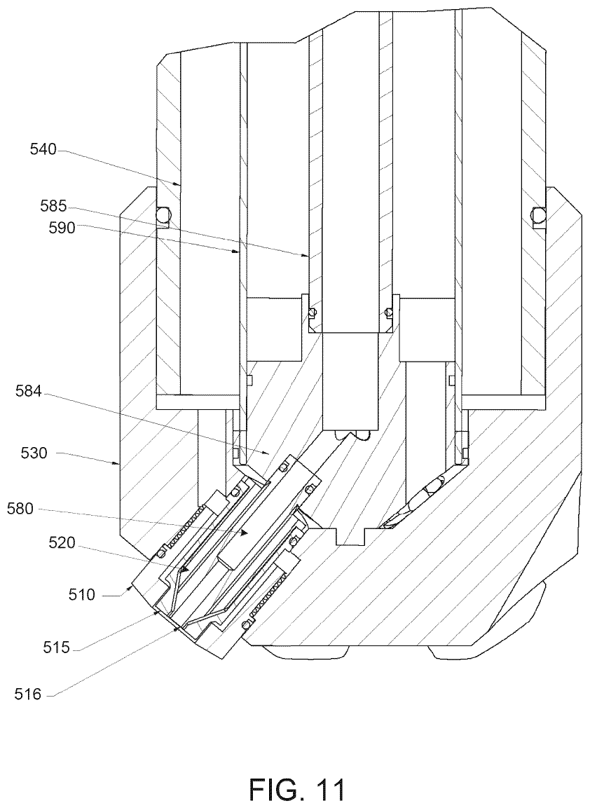

Turning to FIG. 11, an illustrative multi-head assembly is provided in cross-section to show details of one of multiple spray nozzles incorporated into a multi-nozzle assembly. In the illustrative example, a fluid tip 580 (grounded) receives feedstock fluid from a feedstock delivery manifold 584 that is fed by a fluid tube 585 that are also grounded to form the second (grounded) conductive path through which feedstock passes prior to atomizing and expulsion from one of multiple openings (such as opening 515) of the multiple spray nozzle apertures (such as aperture 516 proximate an induction ring 510). Similar to the single nozzle arrangement described above, a plurality of threaded receptacles are provided (one for each induction) in a nozzle head 530 (electrically connected to the induction ring 510) for holding a corresponding induction ring (e.g. induction ring 510) and providing a part of the first conductive path from the induction rings to the electrical induction field source described herein above with reference to a single spray nozzle configuration. The nozzle head 530 is electrically connected to a purge gas tube 540 providing a further segment of the first conductive path.

With continued reference to FIG. 11, an atomizing gas cap 520 and an atomizing gas tube 290 provide an electrical insulating barrier between the first conductive path and the second conductive path described herein above with reference to the illustrative multi-spray nozzle head structure in accordance with the current disclosure.

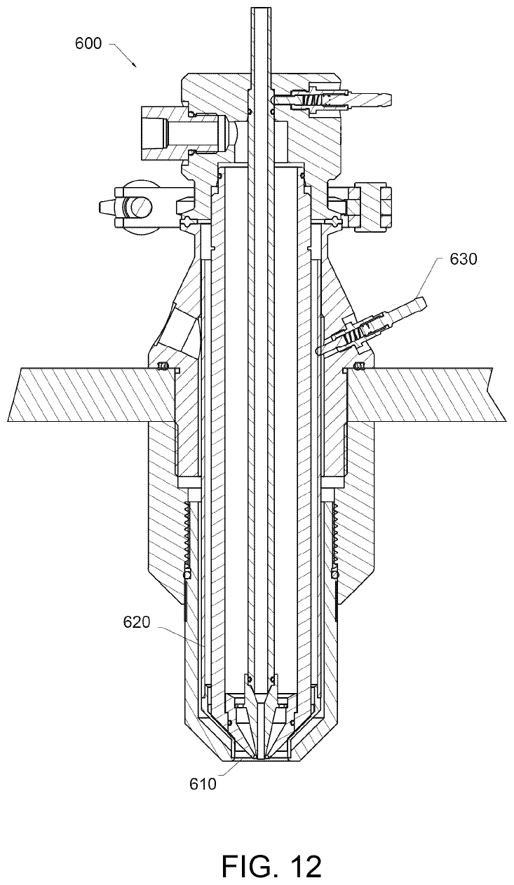

Turning to FIG. 12, a further cross-sectional view of an electrostatic spray nozzle assembly 600 in accordance with a further provided illustrative example. In the illustrative alternative arrangement, the second conductive path (traversed by the feed stock and electrically coupled to electrical ground) and insulating barrier between the second conductive path and the first conductive path are substantially the same as the illustrative example provided in FIG. 8.

However, in the illustrative example provided by FIG. 12 conductive components of the first conductive path are physically shielded, by nozzle mount/shell components, from an environment external to the electrostatic spray nozzle 600. In the illustrative example, an air atomizer is provided inside the circumference of the induction ring 610. A purge air stream is provided by a stainless steel tube 620 connecting the induction ring 610 to a high (magnitude) voltage source (e.g. at electrode 630). In the illustrative example, the air atomizer component may be interchanged with any of a variety of nozzles including: a purely hydraulic nozzle, an internal mixing air atomizer, an ultrasonic atomizer, etc. Importantly, the nozzle mount/shell components provide a physical and electrically insulating barrier between the induction ring 610 and a chamber containing dust created by the dried feed stock.

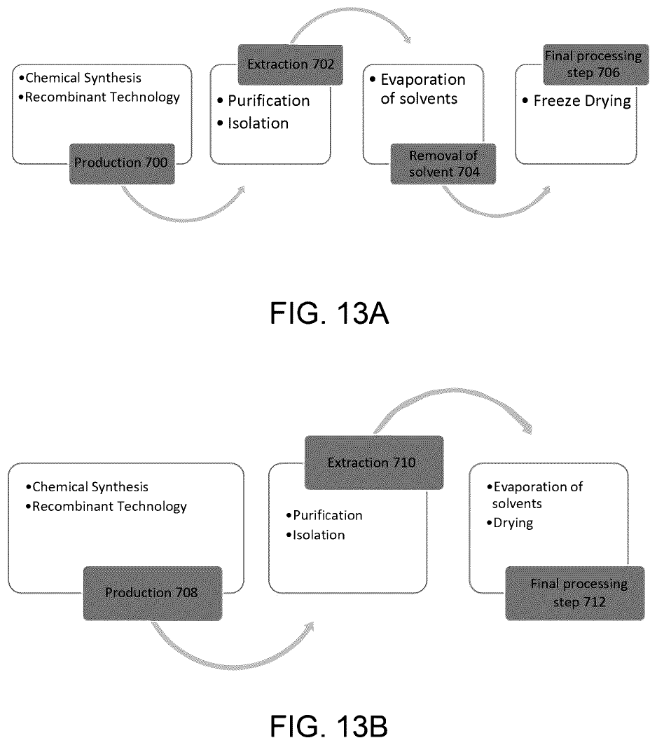

FIG. 13A is a flow diagram depicting an illustrative example of a conventional spray drying process comprising four steps: (1) a production step 700, (2) an extraction step 702, (3) a removal of solvent step 704, and (4) a final processing step 706. FIG. 13B is a flow diagram depicting an illustrative example of techniques in accordance with the present disclosure. As seen in FIG. 13B, to illustrate, three steps can be performed: (1) an illustrative (but not limiting) production step 708, (2) an extraction step 710, and (3) a final processing step 712. In contradistinction to the conventional technique depicted in FIG. 13A, the techniques of the present disclosure can reduce the number of necessary steps, thereby promoting efficiency for the process in accordance with preferred embodiments.

Furthermore, while the illustrative examples have been depicted and described with reference to an exemplary electrostatic spray nozzle assembly configurations, the disclosure is not limited to such assemblies. It will be readily appreciated that, in view of the current disclosure, the advantages of the current disclosure are also applicable to a variety of electrostatic spraying systems that include an induction ring. As such, the current disclosure is intended to apply to a wide variety of electrostatic spray nozzle arrangements—with appropriate adjustments to the above-described structures to accommodate variations in particular electrostatic spray applications.

The disclosure is further illustrated by the following exemplary aspects. However, the disclosure is not limited by the following aspects.
(1) Methods of drying a therapeutic powder, as described herein.
(2) A method of drying a therapeutic powder, the method comprising: providing a feed solution containing a purified therapeutic agent that is extracted into a solvent; electrostatically spray drying the feed solution containing the purified therapeutic agent to remove the solvent to form the therapeutic powder.
(3) The method of aspect 2, wherein the therapeutic agent includes peptides, enzymes, nucleotides, oligonucleotides, gene therapy agents, vaccines, and/or oligonucleotides.
(4) The method of aspect 2 or 3, wherein the therapeutic agent is a peptide.
(5) The method of aspect 4, wherein the peptide functions as a hormone, a growth factor, a neurotransmitter, an ion channel ligand, or an anti-infective agent.
(6) The method of aspect 5, wherein the anti-infective agent is an antiviral agent.
(7) The method of aspect 5, wherein the anti-infective agent is an antibiotic agent.
(8) The method of any one of aspects 2-7, wherein the therapeutic agent comprises a glucagon-like peptide 1 (GLP-1).
(9) The method of aspect 8, wherein the peptide is semaglutide, tirzepatide, dulaglutide, dapiglutide, exenatide, survodutide, liraglutide, albiglutide, lixisenatide, GSBR-1290, and/or any combination thereof.
(10) The method of any one of aspects 2-7, wherein the therapeutic agent comprises a sodium glucose cotransporter 2 (SGLT-2) inhibitor.
(11) The method of aspect 10, wherein the SGLT-2 inhibitor is canagliflozin, ertugliflozin, bexaglifloxin, dapagliflozin, empagliflozin, and/or any combination thereof.
(12) The method of any one of aspects 2-11, wherein the therapeutic agent comprises an amylin analog.
(13) The method of aspect 12, wherein the amylin analog is pramlintide, amycretin, cagrilinitide, petrelintide, GUB014295, and/or ACCG-2671.
(14) The method of any one of aspects 2-13, wherein the purification extraction is performed using chromatography.
(15) The method of any one of aspects 2-14, wherein the atomizing temperature is from 40° C. to 150° C., such as from, e.g., 40° C. to 140° C., 40° C. to 130° C., 40° C. to 120° C., 40° C. to 110° C., 40° C. to 100° C., 40° C. to 90° C., 40° C. to 80° C., 40° C. to 70° C., 40° C. to 60° C., 40° C. to 50° C., 50° C. to 150° C., 60° C. to 150° C., 70° C. to 150° C., 80° C. to 150° C., 90° C. to 150° C., 100° C. to 150° C., 110° C. to 150° C., 120° C. to 150° C., 130° C. to 150° C., or 140° C. to 150° C.
(16) The method of any one of aspects 2-15, wherein the feed solution comprises at least 0.5 wt. % therapeutic agent in solvent solution.
(17) The method of any one of aspects 2-16, wherein the solvent contains a Class I solvent according to FDA guidelines.
(18) The method of aspect 17, wherein the Class I solvent contains benzene, carbon tetrachloride, 1,2-dichloroethane, 1,1-dichloroethene, and/or 1,1,1-trichloroethane.
(19) The method of any one of aspects 2-18, wherein the solvent contains a Class II solvent according to FDA guidelines.
(20) The method of aspect 19, wherein the Class II solvent contains acetonitrile, chlorobenzene, chloroform, cyclohexane, 1,2-dichloroethene, 1,2-dimethoxyethane, n,n-dimethylacetamide, n,n-dimethylformamide, 1,4-dioxane, 2-ethoxyethanol, ethylene glycol, formamide, hexane, methanol, 2-methoxyethanol, methylbutylketone, methylcyclohexane, methylene chloride, n-methylpyrrolidone, nitromethane, pyridine, sulfolane, tetrahydrofuran, tetralin, toluene, trichloroethylene, and/or xylene.
(21) The method of any one of aspects 2-20, wherein the solvent contains a Class III solvent according to FDA guidelines.
(22) The method of aspect 21, wherein the Class III solvent contains acetic acid, heptane, acetone, isobutyl acetate, anisole, isopropyl acetate, 1-butanol, methyl acetate, 2-butanol, 3-methyl-1-butanol, butyl acetate, methylethylketone, tert-butylmethyl ether, methylisobutylketone, cumene, 2-methyl-1-propanol, dimethyl sulfoxide, pentane ethanol, 1-pentanol, ethyl acetate, 1-propanol, ethyl ether, 2-propanol, ethyl formate, propyl acetate, and/or formic acid.
(23) The method of any one of aspects 2-22, wherein the solvent comprises water, ethanol, acetaldehyde, benzene, carbon tetrachloride, 1,2-dichloroethane, 1,1-dichloroethene, 1,1,1-trichloroethane, acetonitrile, chlorobenzene, chloroform, cyclohexane, 1,2-dichloroethene, 1,2-dimethoxyethane, n,n-dimethylacetamide, n,n-dimethylformamide, 1,4-dioxane, 2-ethoxyethanol, ethylene glycol, formamide, hexane, methanol, 2-methoxyethanol, methylbutylketone, methylcyclohexane, methylene chloride, n-methylpyrrolidone, nitromethane, pyridine, sulfolane, tetrahydrofuran, tetralin, toluene, trichloroethylene, xylene, acetic acid, heptane, acetone, isobutyl acetate, anisole, isopropyl acetate, 1-butanol, methyl acetate, 2-butanol, 3-methyl-1-butanol, butyl acetate, methylethylketone, tert-butylmethyl ether, methylisobutylketone, cumene, 2-methyl-1-propanol, dimethyl sulfoxide, pentane ethanol, 1-pentanol, ethyl acetate, 1-propanol, ethyl ether, 2-propanol, ethyl formate, propyl acetate, and/or formic acid.
(24) The method of any one of aspects 2-23, wherein the solvent comprises acetonitrile and water.
(25) The method of any one of aspects 2-24, wherein the solvent has a viscosity of from 1 cP to 1500 cP, such as, e.g., from 25 cP to 1500 cP, from 50 cP to 1500 cP, from 75 cP to 1500 cP, from 100 cP to 1500 cP, from 125 cP to 1500 cP, from 150 cP to 1500 cP, from 175 cP to 1500 cP, from 200 cP to 1500 cP, from 225 cP to 1500 cP, from 250 cP to 1500 cP, from 275 cP to 1500 cP, from 300 cP to 1500 cP, from 325 cP to 1500 cP, from 350 cP to 1500 cP, from 375 cP to 1500 cP, from 400 cP to 1500 cP, from 425 cP to 1500 cP, from 450 cP to 1500 cP, from 475 cP to 1500 cP, from 500 cP to 1500 cP, from 600 cP to 1500 cP, from 700 cP to 1500 cP, from 800 cP to 1500 cP, from 900 cP to 1500 cP, from 1000 cP to 1500 cP, from 1100 cP to 1500 cP, from 1200 cP to 1500 cP, from 1300 cP to 1500 cP, from 1400 cP to 1500 cP, from 1 cP to 1400 cP, from 1 cP to 1300 cP, from 1 cP to 1200 cP, from 1 cP to 1100 cP, from 1 cP to 1000 cP, from 1 cP to 900 cP, from 1 cP to 800 cP, from 1 cP to 700 cP, from 1 cP to 600 cP, from 1 cP to 500 cP, from 1 cP to 475 cP, from 1 cP to 450 cP, from 1 cP to 425 cP, from 1 cP to 400 cP, from 1 cP to 375 cP, from 1 cP to 350 cP, from 1 cP to 325 cP, from 1 cP to 300 cP, from 1 cP to 275 cP, from 1 cP to 250 cP, from 1 cP to 225 cP, from 1 cP to 200 cP, from 1 cP to 175 cP, from 1 cP to 150 cP, from 1 cP to 125 cP, from 1 cP to 100 cP, from 1 cP to 75 cP, from 1 cP to 50 cP, or from 1 cP to 25 cP.
(26) The method of any one of aspects 2-25, wherein the feed solution is electrostatically spray dried at an inlet temperature of 140° C. or less, e.g., 130° C. or less, 120° C. or less, 110° C. or less, 100° C. or less, 90° C. or less, 80° C. or less, 70° C. or less, 60° C. or less, 50° C. or less, 40° C. or less, 30° C. or less, 20° C. or less, or 10° C. or less.
(27) The method of any one of aspects 2-26, wherein the electrostatic spray drying uses applied voltage of at least −30 kV, e.g., −30 kV to 30 kV, −30 kV to 20 kV, −30 kV to 15 kV, −30 kV to 10 kV, −30 kV to 5 kV, −30 kV to 0 kV, −30 kV to −5 kV, −30 kV to −10 kV, −30 kV to −15 kV, −30 kV to −20 kV, −30 kV to −25 kV, −25 kV to 30 kV, −20 kV to 30 kV, −15 kV to 30 kV, −10 kV to 30 kV, −5 kV to 30 kV, 0 kV to 30 kV, 5 kV to 30 kV, 10 kV to 30 kV, 15 kV to 30 kV, 20 kV to 30 kV, 25 kV to 30 kV, −25 kV to 0 kV, −20 kV to 0 kV, −15 kV to 0 kV, −10 kV to 0 kV, or −5 kV to 0 kV.
(28) The method of any one of aspects 2-27, wherein the applied voltage is modulated between two or more different voltages.
(29) The method of any one of aspects 2-28, wherein the applied voltage alternates charges.
(30) The method of any one of aspects 2-29, wherein the method further comprises formulating the powder into an injectable dosage form.
(31) The method of aspect 30, wherein the therapeutic agent is a GLP-1.
(32) The method of any one of aspects 2-31, wherein the method further comprises pressing the powder into the form of a tablet.
(33) The method of any one of aspects 2-31, wherein the powder is mixed with a gel in the form of a capsule.
(34) The method of any one of aspects 2-33, wherein the solvent can be at least partially recovered from the electrostatic spray drying process.
(35) The method of aspects 2-34, wherein the spray drying has a residence time of from about 600 sec to 3600 sec, such as, e.g., from 600 sec to 1800 sec, or 1800 sec to 3600 sec.
(36) The method of aspects 2-35, wherein the spray drying comprises introducing the feed solution into a drying chamber comprising a liner and a liner shaking device for imparting shaking movement into the liner.
(37) A therapeutic powder prepared according to any one of aspects 2-36.

It shall be noted that the preceding aspects are illustrative and not limiting. Other exemplary combinations are apparent from the entirety of the description herein. It will also be understood by one of ordinary skill in the art that various embodiments may be used in various combinations with the other embodiments provided herein.

The following example further illustrates the disclosure but, of course, should not be construed as in any way limiting its scope.

### Example 1

This example demonstrates laboratory scale testing of residual acetonitrile (ACN) in bovine serum albumin (BSA) powders after electrostatic drying. Residual ACN retained in BSA powders was analyzed per United States Pharmacopoeia (USP) 467 method, a headspace gas chromatographic method for the determination of residual solvents in pharmaceutical products, active ingredients, and excipients. This method is a procedure for identifying and quantifying any residual solvent present in pharmaceuticals and excipients as a result of production processes.

A feed solution of peptide surrogate (BSA) in an aqueous to solvent mixture of 50:50 ratio of water to acetonitrile was used for all laboratory scale experiments. The temperature of the feed solution was maintained constant as temperature of feed material has an indirect effect on retention of ACN by the BSA powders.

Electrostatic drying was carried out in a PolarDry Model 001. Nitrogen gas was used as atomizing and drying gas. A two fluid nozzle was used for atomizing of the feed solution.

Table 1 demonstrates the lower and upper limits for process conditions and parameters for the laboratory scale testing.

TABLE 1

| Parameter | Lower Limit | Upper limit |
|---|---|---|
| Feed-rate gm/min | 8.7 | 9 |
| Inlet temperature ° C. | 80 | 90 |
| Outlet temperature ° C. | 38.9 | 44.1 |
| Product temperature ° C. | 41.2 | 48.3 |
| Drying gas flow Nm³/hr | 30 | 30 |
| Atomizing gas pressure (kPa) | 380 | 380 |
| Atomizing gas temp ° C. | 45 | 45 |
| Voltage | −15 | −15 |

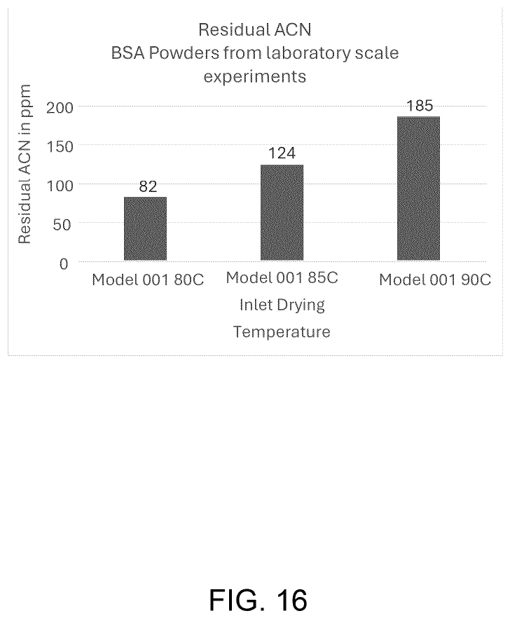

FIG. 16 depicts the laboratory scale results of the residual ACN in BSA powders after electrostatic drying. In FIG. 16, residual ACN (measured in parts per million) is charted along the Y-axis versus inlet drying temperature (measured in ° C.) charted along the X-axis. As seen in FIG. 16, the inventors discovered that, surprisingly and unexpectedly, solvents were efficiently removed at laboratory scale and residual ACN retained in powders was below the ICH limits (<410 ppm).

### Example 2

This example demonstrates production scale testing of the residual acetonitrile (ACN) in bovine serum albumin (BSA), which is a peptide surrogate, powders after electrostatic drying. Residual ACN retained in BSA powders was analyzed per USP 467 method.

A feed solution of peptide surrogate (BSA) in an aqueous to solvent mixture of 50:50 ratio of water to acetonitrile was used for all production scale experiments.

Electrostatic drying was carried out in a PolarDry Model 004 and Model 032. Nitrogen gas was used as atomizing and drying gas. A two fluid nozzle was used for atomizing of the feed solution. Process conditions from laboratory scale experiment as set forth in Table 1 were used to establish parameters for production scale experiments.

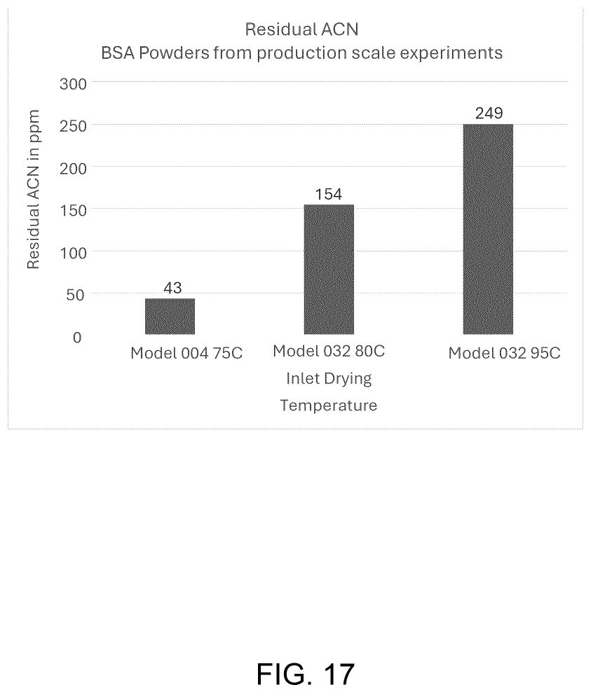

FIG. 17 depicts the production scale results of the residual ACN in BSA powders after electrostatic drying. In FIG. 17, residual ACN (measured in parts per million) is charted along the Y-axis versus inlet drying temperature (measured in ° C.) charted along the X-axis. As seen in FIG. 17, the inventors discovered that, surprisingly and unexpectedly, solvents were efficiently removed at production scale and residual ACN retained in powders was below the ICH limits (<410 ppm).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing embodiments of the disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the embodiments of the disclosure unless otherwise claimed. No language in the specification should be construed as indicating any non-claimed element as essential to the practice of the disclosure.

Preferred embodiments of this disclosure are described herein, including the best mode known to the inventors for carrying out embodiments of the disclosure. All amounts are by weight and not by volume, unless otherwise indicated. Variations of those preferred embodiments may become apparent to those of ordinary skill in the art upon reading the foregoing description. The inventors expect skilled artisans to employ such variations as appropriate, and the inventors intend for embodiments of the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A method of drying a therapeutic powder, the method comprising:
providing a feed solution containing a purified therapeutic agent that is extracted into a solvent;
electrostatically spray drying the feed solution containing the purified therapeutic agent to remove the solvent to form the therapeutic powder.

2. The method of claim 1, wherein the therapeutic agent includes peptides, enzymes, nucleotides, oligonucleotides, gene therapy agents, vaccines, and/or oligonucleotides.

3. The method of claim 2, wherein the therapeutic agent is a peptide.

4. The method of claim 3, wherein the peptide functions as a hormone, a growth factor, a neurotransmitter, an ion channel ligand, or an anti-infective agent.

5. The method of claim 4, wherein the anti-infective agent is an antiviral agent, an antibiotic agent, or a combination thereof.

6. The method of claim 1, wherein the therapeutic agent comprises a glucagon-like peptide 1 (GLP-1), a sodium glucose cotransporter 2 (SGLT-2) inhibitor, amylin analog, or any combination thereof.

7. The method of claim 1, wherein the purification extraction is performed using chromatography.

8. The method of claim 1, wherein the atomizing temperature is from 40° C. to 150° C.

9. The method of claim 1, wherein the solvent contains a Class I solvent according to FDA guidelines, comprising benzene, carbon tetrachloride, 1,2-dichloroethane, 1,1-dichloroethene, and/or 1,1,1-trichloroethane.

10. The method of claim 1, wherein the solvent contains a Class II solvent according to FDA guidelines, comprising acetonitrile, chlorobenzene, chloroform, cyclohexane, 1,2-dichloroethene, 1,2-dimethoxyethane, n,n-dimethylacetamide, n,n-dimethylformamide, 1,4-dioxane, 2-ethoxyethanol, ethylene glycol, formamide, hexane, methanol, 2-methoxyethanol, methylbutylketone, methylcyclohexane, methylene chloride, n-methylpyrrolidone, nitromethane, pyridine, sulfolane, tetrahydrofuran, tetralin, toluene, trichloroethylene, and/or xylene.

11. The method of claim 1, wherein the solvent contains a Class III solvent according to FDA guidelines, comprising acetic acid, heptane, acetone, isobutyl acetate, anisole, isopropyl acetate, 1-butanol, methyl acetate, 2-butanol, 3-methyl-1-butanol, butyl acetate, methylethylketone, tert-butylmethyl ether, methylisobutylketone, cumene, 2-methyl-1-propanol, dimethyl sulfoxide, pentane ethanol, 1-pentanol, ethyl acetate, 1-propanol, ethyl ether, 2-propanol, ethyl formate, propyl acetate, and/or formic acid.

12. The method of claim 1, wherein the solvent comprises water, ethanol, acetaldehyde, benzene, carbon tetrachloride, 1,2-dichloroethane, 1,1-dichloroethene, 1,1,1-trichloroethane, acetonitrile, chlorobenzene, chloroform, cyclohexane, 1,2-dichloroethene, 1,2-dimethoxyethane, n,n-dimethylacetamide, n,n-dimethylformamide, 1,4-dioxane, 2-ethoxyethanol, ethylene glycol, formamide, hexane, methanol, 2-methoxyethanol, methylbutylketone, methylcyclohexane, methylene chloride, n-methylpyrrolidone, nitromethane, pyridine, sulfolane, tetrahydrofuran, tetralin, toluene, trichloroethylene, xylene, acetic acid, heptane, acetone, isobutyl acetate, anisole, isopropyl acetate, 1-butanol, methyl acetate, 2-butanol, 3-methyl-1-butanol, butyl acetate, methylethylketone, tert-butylmethyl ether, methylisobutylketone, cumene, 2-methyl-1-propanol, dimethyl sulfoxide, pentane ethanol, 1-pentanol, ethyl acetate, 1-propanol, ethyl ether, 2-propanol, ethyl formate, propyl acetate, and/or formic acid.

13. The method of claim 1, wherein the solvent comprises acetonitrile and water.

14. The method of claim 1, wherein the feed solution is electrostatically spray dried at an inlet temperature of 140° C. or less.

15. The method of claim 1, wherein the electrostatic spray drying uses applied voltage of at least −30 kV.

16. The method of claim 17, wherein the applied voltage is modulated between two or more different voltages.

17. The method of claim 17, wherein the applied voltage alternates charges.

18. The method of claim 1, wherein the solvent can be at least partially recovered from the electrostatic spray drying process.

19. The method of claim 1, wherein the spray drying has a residence time of from about 600 sec to 3600 sec.

20. The method of claim 1, wherein the spray drying comprises introducing the feed solution into a drying chamber comprising a liner and a liner shaking device for imparting shaking movement into the liner.
